SEQUENCE LISTING (84 sequences)

SEQ ID NO: 1 - DNA, 17 bp, synthetic construct
  source 1..17: mol_type = other DNA
ggaaggacggttttttg

SEQ ID NO: 2 - DNA, 18 bp, synthetic construct
  source 1..18: mol_type = other DNA
ggaaggatggttttttgg

SEQ ID NO: 3 - DNA, 17 bp, synthetic construct
  source 1..17: mol_type = other DNA
atgatttatagggttgg

SEQ ID NO: 4 - DNA, 14 bp, synthetic construct
  source 1..14: mol_type = other DNA
aacttccccaacac

SEQ ID NO: 5 - DNA, 15 bp, synthetic construct
  source 1..15: mol_type = other DNA
cgcagcgtggtgagc

SEQ ID NO: 6 - DNA, 15 bp, synthetic construct
  source 1..15: mol_type = other DNA
cgcagcatggtgagc

SEQ ID NO: 7 - DNA, 17 bp, synthetic construct
  source 1..17: mol_type = other DNA
ggctgtgggttggtgat

SEQ ID NO: 8 - DNA, 19 bp, synthetic construct
  source 1..19: mol_type = other DNA
catagtggtcagcaggcag

SEQ ID NO: 9 - DNA, 19 bp, synthetic construct
  source 1..19: mol_type = other DNA
ccacatacccggaaaccac

SEQ ID NO: 10 - DNA, 19 bp, synthetic construct
  source 1..19: mol_type = other DNA
ccacatacccagaaaccac

SEQ ID NO: 11 - DNA, 20 bp, synthetic construct
  source 1..20: mol_type = other DNA
ccctcatctccatcttctgc

SEQ ID NO: 12 - DNA, 21 bp, synthetic construct
  source 1..21: mol_type = other DNA
  modified_base 5: mod_base = OTHER; note = Inosine
gcacnatgagacagttaaagg

SEQ ID NO: 13 - DNA, 17 bp, synthetic construct
  source 1..17: mol_type = other DNA
aggtgccggactgtagg

SEQ ID NO: 14 - DNA, 17 bp, synthetic construct
  source 1..17: mol_type = other DNA
aggtgccagactgtagg

SEQ ID NO: 15 - DNA, 17 bp, synthetic construct
  source 1..17: mol_type = other DNA
  modified_base 2: mod_base = OTHER; note = Inosine
cngaagcaggccctaag

SEQ ID NO: 16 - DNA, 20 bp, synthetic construct
  source 1..20: mol_type = other DNA
cttcaggtagggcttctcag

SEQ ID NO: 17 - DNA, 17 bp, synthetic construct
  source 1..17: mol_type = other DNA
tgagcccggcatgttga

SEQ ID NO: 18 - DNA, 17 bp, synthetic construct
  source 1..17: mol_type = other DNA
tgagcccagcatgttga

SEQ ID NO: 19 - DNA, 18 bp, synthetic construct
  source 1..18: mol_type = other DNA
  modified_base 17: mod_base = OTHER; note = Inosine
gctagaatgcagtgacnc

SEQ ID NO: 20 - DNA, 18 bp, synthetic construct
  source 1..18: mol_type = other DNA
  modified_base 8: mod_base = OTHER; note = Inosine
cacagtcnctcacctctg

SEQ ID NO: 21 - DNA, 20 bp, synthetic construct
  source 1..20: mol_type = other DNA
tctaagtgccgcagactgaa

SEQ ID NO: 22 - DNA, 20 bp, synthetic construct
  source 1..20: mol_type = other DNA
tctaagtgccacagactgaa

SEQ ID NO: 23 - DNA, 20 bp, synthetic construct
  source 1..20: mol_type = other DNA
aatattgagcttccccaggc

SEQ ID NO: 24 - DNA, 20 bp, synthetic construct
  source 1..20: mol_type = other DNA
  modified_base 18: mod_base = OTHER; note = Inosine
actcctgactaatagccntg

SEQ ID NO: 25 - DNA, 18 bp, synthetic construct
  source 1..18: mol_type = other DNA
tgatgtgttcgcagcagt

SEQ ID NO: 26 - DNA, 18 bp, synthetic construct
  source 1..18: mol_type = other DNA
tgatgtgttcacagcagt

SEQ ID NO: 27 - DNA, 21 bp, synthetic construct
  source 1..21: mol_type = other DNA
gagagagcatgacagaagaga

SEQ ID NO: 28 - DNA, 23 bp, synthetic construct
  source 1..23: mol_type = other DNA
  modified_base 12: mod_base = OTHER; note = Inosine
tcactttgaacnttatgaacttc

SEQ ID NO: 29 - DNA, 18 bp, synthetic construct
  source 1..18: mol_type = other DNA
cccttccccgtaggatat

SEQ ID NO: 30 - DNA, 18 bp, synthetic construct
  source 1..18: mol_type = other DNA
cccttccccataggatat

SEQ ID NO: 31 - DNA, 20 bp, synthetic construct
  source 1..20: mol_type = other DNA
catgcactcaacacacacac

SEQ ID NO: 32 - DNA, 18 bp, synthetic construct
  source 1..18: mol_type = other DNA
gggaggatggcagtagga

SEQ ID NO: 33 - DNA, 18 bp, synthetic construct
  source 1..18: mol_type = other DNA
ttgagctcgagcctcatg

SEQ ID NO: 34 - DNA, 18 bp, synthetic construct
  source 1..18: mol_type = other DNA
ttgagctcaagcctcatg

SEQ ID NO: 35 - DNA, 20 bp, synthetic construct
  source 1..20: mol_type = other DNA
  modified_base 7: mod_base = OTHER; note = Inosine
ggtatcntgcttagctgtct

SEQ ID NO: 36 - DNA, 17 bp, synthetic construct
  source 1..17: mol_type = other DNA
  modified_base 17: mod_base = OTHER; note = Inosine
atcctggatctgcaccn

SEQ ID NO: 37 - DNA, 17 bp, synthetic construct
  source 1..17: mol_type = other DNA
cagccgtaagtgcagag

SEQ ID NO: 38 - DNA, 17 bp, synthetic construct
  source 1..17: mol_type = other DNA
cagccataagtgcagag

SEQ ID NO: 39 - DNA, 22 bp, synthetic construct
  source 1..22: mol_type = other DNA
cactctgcttgtttctatcacc

SEQ ID NO: 40 - DNA, 18 bp, synthetic construct
  source 1..18: mol_type = other DNA
  modified_base 13: mod_base = OTHER; note = Inosine
cagtgaaagggcngagtc

SEQ ID NO: 41 - DNA, 17 bp, synthetic construct
  source 1..17: mol_type = other DNA
gttacgcaacacgactg

SEQ ID NO: 42 - DNA, 17 bp, synthetic construct
  source 1..17: mol_type = other DNA
gttacacaacacgactg

SEQ ID NO: 43 - DNA, 20 bp, synthetic construct
  source 1..20: mol_type = other DNA
gcctgaagaccacattctga

SEQ ID NO: 44 - DNA, 20 bp, synthetic construct
  source 1..20: mol_type = other DNA
acacacaagctgtctggtaa

SEQ ID NO: 45 - DNA, 18 bp, synthetic construct
  source 1..18: mol_type = other DNA
caggcacgtaagctgttc

SEQ ID NO: 46 - DNA, 18 bp, synthetic construct
  source 1..18: mol_type = other DNA
caggcacataagctgttc

SEQ ID NO: 47 - DNA, 20 bp, synthetic construct
  source 1..20: mol_type = other DNA
  modified_base 4: mod_base = OTHER; note = Inosine
cccntttctcttctgatcca

SEQ ID NO: 48 - DNA, 20 bp, synthetic construct
  source 1..20: mol_type = other DNA
agctcagaaccccacagata

SEQ ID NO: 49 - DNA, 18 bp, synthetic construct
  source 1..18: mol_type = other DNA
accttgtgcgtttgttgc

SEQ ID NO: 50 - DNA, 18 bp, synthetic construct
  source 1..18: mol_type = other DNA
accttgtgcatttgttgc

SEQ ID NO: 51 - DNA, 20 bp, synthetic construct
  source 1..20: mol_type = other DNA
aagggttttctttgaaggcc

SEQ ID NO: 52 - DNA, 22 bp, synthetic construct
  source 1..22: mol_type = other DNA
ctgtgcctgtgtcatttactat

SEQ ID NO: 53 - DNA, 17 bp, synthetic construct
  source 1..17: mol_type = other DNA
aacagttcgaatgcgcc

SEQ ID NO: 54 - DNA, 17 bp, synthetic construct
  source 1..17: mol_type = other DNA
aacagttcaaatgcgcc

SEQ ID NO: 55 - DNA, 19 bp, synthetic construct
  source 1..19: mol_type = other DNA
cacagcatcagagggtctc

SEQ ID NO: 56 - DNA, 20 bp, synthetic construct
  source 1..20: mol_type = other DNA
  modified_base 15: mod_base = OTHER; note = Inosine
tcaaaggtcagttcnctctt

SEQ ID NO: 57 - DNA, 17 bp, synthetic construct
  source 1..17: mol_type = other DNA
caacctgcggtgagtgg

SEQ ID NO: 58 - DNA, 17 bp, synthetic construct
  source 1..17: mol_type = other DNA
caacctgcagtgagtgg

SEQ ID NO: 59 - DNA, 17 bp, synthetic construct
  source 1..17: mol_type = other DNA
  modified_base 12: mod_base = OTHER; note = Inosine
agaggctgagcnaggac

SEQ ID NO: 60 - DNA, 18 bp, synthetic construct
  source 1..18: mol_type = other DNA
ctcattcctgggctccac

SEQ ID NO: 61 - DNA, 16 bp, synthetic construct
  source 1..16: mol_type = other DNA
ccagggcgtctcgaat

SEQ ID NO: 62 - DNA, 16 bp, synthetic construct
  source 1..16: mol_type = other DNA
ccagggcatctcgaat

SEQ ID NO: 63 - DNA, 20 bp, synthetic construct
  source 1..20: mol_type = other DNA
aaagaactggcttacttggc

SEQ ID NO: 64 - DNA, 20 bp, synthetic construct
  source 1..20: mol_type = other DNA
gcctcttgtgttccctagaa

SEQ ID NO: 65 - DNA, 16 bp, synthetic construct
  source 1..16: mol_type = other DNA
caaaccgcccccagag

SEQ ID NO: 66 - DNA, 16 bp, synthetic construct
  source 1..16: mol_type = other DNA
caaaccacccccagag

SEQ ID NO: 67 - DNA, 20 bp, synthetic construct
  source 1..20: mol_type = other DNA
gatcttgcccatattgctgc

SEQ ID NO: 68 - DNA, 23 bp, synthetic construct
  source 1..23: mol_type = other DNA
cagattccagagactaaacttgt

SEQ ID NO: 69 - DNA, 19 bp, synthetic construct
  source 1..19: mol_type = other DNA
cacttatcgtgccaaactc

SEQ ID NO: 70 - DNA, 19 bp, synthetic construct
  source 1..19: mol_type = other DNA
cacttatcatgccaaactc

SEQ ID NO: 71 - DNA, 20 bp, synthetic construct
  source 1..20: mol_type = other DNA
  modified_base 15: mod_base = OTHER; note = Inosine
cattcaatggcagcngttag

SEQ ID NO: 72 - DNA, 20 bp, synthetic construct
  source 1..20: mol_type = other DNA
ttgctgttctcttccacctc

SEQ ID NO: 73 - DNA, 16 bp, synthetic construct
  source 1..16: mol_type = other DNA
ccagggcgggaggaag

SEQ ID NO: 74 - DNA, 16 bp, synthetic construct
  source 1..16: mol_type = other DNA
ccagggcaggaggaag

SEQ ID NO: 75 - DNA, 20 bp, synthetic construct
  source 1..20: mol_type = other DNA
tctaacaccagcagcagaaa

SEQ ID NO: 76 - DNA, 20 bp, synthetic construct
  source 1..20: mol_type = other DNA
tggaagtagaaagtggtgct

SEQ ID NO: 77 - DNA, 18 bp, synthetic construct
  source 1..18: mol_type = other DNA
ccaggcccggatagatag

SEQ ID NO: 78 - DNA, 18 bp, synthetic construct
  source 1..18: mol_type = other DNA
ccaggcccagatagatag

SEQ ID NO: 79 - DNA, 20 bp, synthetic construct
  source 1..20: mol_type = other DNA
ccatccctccactctgaatt

SEQ ID NO: 80 - DNA, 20 bp, synthetic construct
  source 1..20: mol_type = other DNA
  modified_base 20: mod_base = OTHER; note = Inosine
tcactaaatggaatgcagcn

SEQ ID NO: 81 - DNA, 18 bp, synthetic construct
  source 1..18: mol_type = other DNA
cttggcctcgaaaaagcc

SEQ ID NO: 82 - DNA, 18 bp, synthetic construct
  source 1..18: mol_type = other DNA
cttggcctcaaaaaagcc

SEQ ID NO: 83 - DNA, 19 bp, synthetic construct
  source 1..19: mol_type = other DNA
  modified_base 18: mod_base = OTHER; note = Inosine
gtcatctggttgagctcna

SEQ ID NO: 84 - DNA, 20 bp, synthetic construct
  source 1..20: mol_type = other DNA
cactgtgacatgcaatcctg

(19) United States
(12) Patent Application Publication
(10) Pub. No.: US 2026/0085357 A1
(43) Pub. Date: Mar. 26, 2026

(54) COMPOSITIONS, SYSTEMS, AND METHODS FOR DETECTION OF OVARIAN CANCER

(71) Applicant: Johnson Yiu-Nam LAU, Houston, TX (US)

(72) Inventors: Manson Fok, Hong Kong (CN); Kang Zhang, Hong Kong (CN)

(21) Appl. No.: 19/114,203

(22) PCT Filed: Sep. 26, 2023
(86) PCT No.: PCT/US2023/033727

§ 371 Date: Mar. 21, 2025

Related U.S. Application Data
(60) Provisional application No. 63/409,986, filed on Sep. 26, 2022.

(51) Int. Cl. C12Q 1/6886; G16B 20/20; G16B 40/20

(52) U.S. Cl. CPC C12Q 1/6886; G16B 20/20; G16B 40/20; C12Q 2600/118; C12Q 2600/156

(57) ABSTRACT

Compositions and methods for diagnosis and evaluation of prognosis of epithelial ovarian cancer (EOC) are provided. Development of an artificial intelligence approach entitled MethylBert and its application to identification of methylation sites useful in identification of EOC from cell free DNA obtained from serum or plasma is shown. PCR-based methods directed to the methylation state of the OV-1 site identified using MethylBERT approach can be used in screening for EOC using cell free DNA obtained from blood samples, and can be combined with measurement of tumor markers associated with EOC.

This application claims the benefit of U.S. Provisional Application No. 63/409,986 filed on Sep. 26, 2022. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is methods for diagnosing cancer, in particular ovarian cancer.

### BACKGROUND

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Ovarian cancer was an important cause of cancer death in women, with an incidence of 313,000 and with over 200 thousand deaths in 2020, 85%-95% of ovarian cancer were from epithelial cells. Although breast and cervical cancers are more common, epithelial ovarian cancer (EOC) has a much lower 5-year survival rate, which makes EOC more lethal to female when compared with breast and cervical cancers. This high mortality and low 5-year survival rate of EOC were mainly related to a late diagnosis, with more than 80% of patients already at advanced stages when diagnosed. Based on current data available, if EOC was diagnosed at stage I, the 5-year survival rate would be at around 90%. This rapidly declines to around 20% if diagnosed at the later stage III/IV.

At present, serum biomarker CA125 and transvaginal ultrasound examination (TVU) are the two most commonly used tests for EOC screening. Serum HE4 has also emerged as an important serum biomarker for EOC diagnosis and is implicated the detection of recurrence. The use of CA125 alone for EOC detection has a low sensitivity, whereas TVU is highly sensitive and accurate for EOC detection, however, routine TVU use for first-line mass EOC screening is clinically not feasible due to its inconvenience, time-consuming, and conclusion largely depends on the experience of sonographer. Nevertheless, two large clinical studies have found that annual evaluation of CA125 alone, or in combination with TVU showed no reduction in EOC-related mortality. These findings highlight the urgent need for a highly sensitive and specific EOC test that is effective for the early detection of EOC.

Circulating cell-free DNA (cfDNA) are extracellular nucleic acid fragments found in liquid biopsies. When cfDNA are shed by tumor cells, for instance during apoptosis, they are potentially useful in the diagnosis of cancer because they contain the same genetic and epigenetic alterations of the tumor cells from which they derive. The potential use of cfDNA in EOC screening has shown some promising results. However, these studies were limited by a small sample size, and the samples were biased towards later stage EOC. Therefore, the utility of these tests for the diagnosis of early EOC was not well characterized. Furthermore, a comprehensive and systematic genome-wide analysis of EOC-specific methylation markers is lacking.

Thus, there is still a need for sensitive and accurate methods for diagnosing ovarian cancer (EOC) using readily-obtainable samples.

### SUMMARY OF THE INVENTION

The inventive subject matter provides apparatus, systems and methods for diagnosing epithelial ovarian cancer (EOC) and/or providing a prognosis for EOC by evaluating the methylation state at one or more sites identified using artificial intelligence screening of cell free DNA (cfDNA) samples provided by healthy individuals and individual with EOC.

Embodiments of the inventive concept include methods of assisting in diagnoses of epithelial ovarian cancer (EOC) by isolating cell-free genetic material from an individual, followed by characterizing nucleic acid methylation of one or more genetic markers from the group of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22. Aberrant methylation of one or more genetic marker is indicative of a diagnosis of epithelial ovarian cancer. In some embodiments the cell-free genetic material is cell-free DNA. Such methods can include a step of treating at least a portion of the cell-free genetic material with bisulfite, and can include steps of contacting the cell-free genetic material with a nucleic acid primer complementary to a portion of the genetic material proximal to at least one of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22 and performing an nucleic acid amplification to generate an amplification product on bisulfite-treated and untreated samples. Such nucleic acid amplification steps can include contacting the resulting amplification product with a probe that is complementary to at least of OV1, OV2, OV6, OV3, OV4, OV5, OV6, OV6, OV6, OV6, OV7, OV8, OV9, OV10, OV11, OV12, OV13, OV14, OV15. OV16, OV17, OV18, OV19, OV20, OV21, and OV22. In some embodiments, such testing is directed to OV1 In some embodiments CA125 is also characterized, wherein abnormally high levels of CA125 are indicative of epithelial ovarian cancer.

Embodiments of the inventive concept include methods of assisting in evaluating the prognosis of an individual with epithelial ovarian cancer by isolating cell-free genetic material from an individual, followed by characterizing nucleic acid methylation of one or more genetic markers selected from OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22. Aberrant methylation of one or more genetic marker is indicative of a poor prognosis and/or advanced disease. In some embodiments the cell-free genetic material is cell-free DNA. Such methods can include a step of treating at least a portion of the cell-free genetic material with bisulfite, and can include steps of contacting the cell-free genetic material with a nucleic acid primer complementary to a portion of the genetic material proximal to at least one of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22 and performing an nucleic acid amplification to generate an amplification product on bisulfite-treated and untreated samples. Such nucleic acid amplification steps can include contacting the resulting amplification product with a probe that is complementary to at least one of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22. In some embodiments, such testing is directed to OV1 In some embodiments CA125 is also characterized, wherein abnormally high levels of CA125 are indicative of a poor prognosis or advanced epithelial ovarian cancer.

Embodiments of the inventive concept include implementation of an artificial intelligence algorithm to identity methylation patterns associated with a diagnosis of EOC and/or prognosis of EOC. Such an artificial intelligence algorithm can be implemented prior to steps of characterizing methylation in methods as described above. In such embodiments, such an artificial intelligence algorithm can include correlation with chromosome embedding, position embedding, methylation level embedding, and gene embedding, and wherein the methylation pattern comprises one or more genetic markers exhibiting methylation differences between individuals with EOC and individuals without EOC. Such an artificial intelligence algorithm can include a matrix decomposition-based Transformer model that reduces computational complexity from quadratic to linear (O(L²) to O(L)), which in turn enables efficient processing of large-scale data. In some embodiments such a Transformer model comprises a Performer, can include an attention mechanism formulated as Att(Q, K, V)=softmax(ΦQ)Φ(K)^{T}/sqrt(dₖ)V, where Q, K, and V denote query, key, and value matrices, respectively, and Φ signifies a feature map function that projects input into a new space. Such a Performer can provide efficient approximation of the dot-product attention. Such an artificial intelligence algorithm can be applied to a training dataset comprising individuals identified as having EOC and individuals without EOC and identifying a methylation pattern associated with individuals with EOC, wherein the artificial intelligence algorithm identifies a methylation pattern that includes one or more genetic markers exhibiting methylation differences between individuals with EOC and individuals without EOC.

Another embodiment of the inventive concept is a composition for diagnosis or prognosis of epithelial ovarian cancer, which includes a first primer that is complementary to a first portion of nucleic acid proximal to a genetic marker that is OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, or OV22. Such a composition also includes and a probe that is complementary to at least one genetic marker that is OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, or OV22 and that comprises a first dye or a first fluorophore. In some embodiments the genetic marker is OV1. In some embodiments the composition includes a second primer that is directed to a second genetic marker that is one of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22. In such embodiments the composition can include a second probe that is complementary to OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, or OV22. In such embodiments the second probe can include a second dye or a second fluorophore.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1: FIG. 1 provides a flowchart depicting identification of methylation sites useful for diagnosis and prognosis of EOC status using LASSO.

FIGS. 2A to 2F: FIG. 2A provides a Confusion table of binary results of the OCDP in the training dataset. FIG. 2B provides a Confusion tables of binary results of the OCDP in the validation dataset. FIG. 2C shows typical ROC curves of the diagnostic prediction model with methylation markers in the training data set. FIG. 2D shows atypical ROC curve of the diagnostic prediction model with methylation markers in the validation data sets. FIG. 2E shows typical unsupervised hierarchical clustering of seven methylation markers selected for use in the diagnostic prediction model. FIG. 2F show typical unsupervised hierarchical clustering of seven methylation markers selected for use in the diagnostic prediction model in the validation data sets.

FIGS. 3A to 3E: FIGS. 3A to 3 show results of studies of the OCDP and CA125 for different stages of EOC. FIG. 3A provides Confusion tables of binary results of the diagnostic prediction model for different cancer stages of ovarian cancer and healthy female. FIG. 3B shows typical ROC curves of the OCDP for distinguishing early stage (red) or advanced stage (blue) EOC patients from healthy female. FIG. 3C shows typical ROC curves of the OCDP (red) and OCDP in combination with CA125 (blue) for distinguishing early stage EOC from healthy female. Note: only samples with CA125 information were summarized in this figure. FIG. 3D depicts CA12 levels of healthy female samples and EOC samples of different stages. FIG. 3E depicts CA12 OCDP cd-score values of healthy female samples and EOC samples of different stages.

FIGS. 4A to 4D: FIGS. 4A to 4D show results of studies of the utility of the OCPP for prognosis prediction of EOC. FIG. 4A shows exemplary Kaplan-Meier plots for overall survival of EOC patients in the low- and high-risk groups determined by the OCPP in the training datasets. FIG. 4B shows typical Kaplan-Meier plots for overall survival of EOC patients in the low- and high-risk groups determined by the OCPP in the validation dataset. FIG. 4C shows typical ROC curves and corresponding AUCs of 5-year survival prediction by OCPP cp score and CA125 and in early EOC. FIG. 4D shows results of multivariable analysis for early EOC survival with covariates including OCPP cp score and CA125.

FIGS. 5A to 5G: FIG. 5E show data related to performance of the ddPCR assay with OV1 in discriminating ovarian cancer and healthy female. FIG. 5A shows typical ROC curves of OV1 in the training (red) and validation (blue) datasets of the ddPCR cohort. FIG. 5B provides a Confusion table of binary results of the OV1 prediction model in the training dataset. FIG. 5C provides a Confusion table of binary results of the OV1 prediction model in the validation datasets. FIG. 5D shows typical ROC curves of OV1 and OV1-CA125 combination in distinguishing early (red and green) and advanced (blue and purple) EOC from healthy female in the ddPCR cohort. FIG. 5E provides a Confusion table summarizing OV1 distinguishing early and advanced EOC. FIG. 5F provides a summary of sensitivity and specificity of CA125, OVA-1 ddPCR, and combined CA125 and OVA-1 PCR results in healthy, early EOC, and advanced EOC. FIG. 5G shows Beeswarm plots presenting the methylation levels of OV1 in the ddPCR cohort between ovarian cancer and healthy female, red plots are healthy female samples and blue plots are EOC samples.

FIG. 6: FIG. 6 provides a flowchart depicting identification of methylation sites useful for diagnosis and prognosis of EOC status using MethylBERT.

FIGS. 7A to 7G: FIGS. 7A to 7G show cfDNA methylation analysis of MethylBERT-EOC diagnosis mode. FIG. 2A provides a schematic overview of MethylBERT model. FIG. 7B provides a Confusion table of binary results of MethylBERT-EOC diagnostic model in the training dataset. FIG. 7C provides a Confusion tables of binary results of MethylBERT-EOC diagnostic model in the validation dataset. FIG. 7D shows typical ROC curves of MethylBERT-EOC diagnostic model in EOC diagnostic prediction the training and validation data sets. FIG. 7E shows a typical ROC curve of MethylBERT-EOC diagnostic model in early and advanced EOC diagnostic prediction in the training dataset. FIG. 7F shows typical ROC curves of MethylBERT-EOC diagnostic model in early and advanced EOC diagnostic prediction in the validation dataset. FIG. 7G provides MethylBERT-EOC diagnosis based EOC prediction score of healthy female samples and EOC samples of different stages.

FIG. 8: FIG. 8 shows typical data from studies characterizing relative sensitivities of CA125 measurement and application of the MethyIBERT model for EOC.

FIGS. 9A to 9F: FIGS. 9A to 9D show results from cfDNA methylation analysis using the LASSO-EOC diagnosis model in the individual cohort. FIG. 9A provides a Confusion table of binary results of the LASSO-EOC diagnostic model in the training dataset. FIG. 9B provides a Confusion table of binary results of the LASSO-EOC diagnostic model in the validation dataset. FIG. 9C shows typical ROC curves of the LASSO-EOC diagnostic model in EOC diagnostic prediction the training and validation data sets. FIG. 9D shows typical ROC curves of the LASSO-based EOC diagnostic model in early and advanced EOC diagnostic prediction the training dataset. FIG. 9E shows typical ROC curves of the LASSO-based EOC diagnostic model in early and advanced EOC diagnostic prediction in the validation dataset. FIG. 9F provides results from LASSO-EOC diagnosis based EOC prediction score of healthy female samples and EOC samples of different stages.

### DETAILED DESCRIPTION

The inventive subject matter provides apparatus, systems and methods in which circulating, cell-free DNA obtained from conveniently obtained serum or plasma samples can be used to diagnose epithelial ovarian cancer (EOC) in a sensitive and accurate manner. Inventors have determined a panel of DNA methylation modifications that can be characterized using suitable methods (e.g., DNA sequencing, hybridization, PCR, etc.) to determine if an individual has early stage (Grade I or II) or late stage (Grade III or IV) epithelial ovarian cancer. The panel is particularly useful in identifying early stage disease, which is more treatable but relatively asymptomatic. Accuracy and sensitivity of such a panel can be improved by incorporating results from assays for cancer markers associated with EOC (e.g., CA-125 and/or HE4).

In addition, Inventors have determined that characterization of methylation at a single site (e.g., at the site designated OV1), for example using DNA amplification using methylation-specific primer and/or probes, can provide a simplified testing method that can provide results that aid in accurate and sensitive diagnosis of EOC at both early and late stages, particularly when paired with assays for cancer markers associated with EOC (e.g., CA125 and/or HE4).

Inventors performed artificial intelligence assisted genome-wide surveys by screening 3.3 million methylation CpG positions to identify a panel of EOC specific methylation markers using cell-free DNA (cfDNA) samples from EOC patients and healthy female subjects.

In an exemplary study Inventors used eight selected methylation markers as a diagnostic panel based on a training dataset of 471 EOC patients and 742 healthy female subjects which showed 86.62% sensitivity on 89.08% specificity (AUC=0.933), and on a validation dataset (283 EOC patients and 376 healthy females) that showed 84.45% sensitivity on 87.77% specificity (AUC=0.926). Importantly, detection rate was high for early-stage EOC (stage I/I) (72.68%, n=205) when compared to all stage EOC patients (85.81%, n=754). By contrast, the sensitivity for conventional CA-125 and HE-4 markers were 48.81% and 46.85%, respectively (combined sensitivity of 60.6%). Moreover, of the 282 EOC patients missed by CA-125/HE4, the panel was able to detect 235 (83.33%) of these patients. The sensitivity of detection was further improved when the cfDNA methylation diagnostic panel was combined with CA125 (from 73.22% to 84.15% for early EOC patients and from 91.01% to 95.51% for advanced-stage EOC of stage III/IV). The potential adaptation of this approach to a cost-effective and time-saving droplet digital PCR (ddPCR) assay was demonstrated with OV1, the most significant methylation site in our panel. Such results document the potential of utilizing cfDNA methylation patterns as a panel for first-line screening assay for early detection and diagnosis of EOC.

Various objects, features, aspects and advantages of the inventive subject matter will become more apparent from the following detailed description of preferred embodiments, along with the accompanying drawing figures in which like numerals represent like components.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

One should appreciate that the disclosed techniques provide many advantageous technical effects including providing sensitive and accurate diagnosis of the presence of early-stage and late-stage epithelial ovarian cancer as well as prognosis of same, using a readily obtainable serum or plasma sample.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Embodiments of the inventive concept include compositions, methods, and systems that utilize a set of DNA methylation markers (OV1, OV2, OV36, OV4, OV5, OV6, OV7, OV8, OV9, OV10, O₁₁, OV12, OV13, OV14, OV15, OV16, OV17, OV18, OV19, OV20, OV21, and/or OV22), a subset of these methylation markers, or a single methylation marker from this panel in assays that have particular utility in diagnosing epithelial ovarian cancer (in both early and late stages) and/or prognosis for same. In preferred embodiments cell-free DNA obtained from the patient is used, such as that obtained from a blood, serum, or plasma sample.

In some embodiments such assays can incorporate steps of splitting a blood, serum, or plasma sample into at least two portions, bisulfite treatment of cfDNA from one of these portions, and selectively amplifying cfDNA from untreated and bisulfite treated samples using primers and/or probes specific for methylation and post-bisulfite treatment at one or more sites wherein differences in methylation between EOC and control groups are associated with EOC (e.g., OV1, OV2, OV36, OV4, OV5, OV6, OV7, OV8, OV9, OV10, O₁₁, OV12, OV13, OV14, OV15, OV16, OV17, OV18, OV19, OV20, OV21, and/or OV22). Results from these studies can be combined with results from assays (e.g., immunoassays) directed to EOC-associated cancer markers (such as CA125 and/or HE4).

Such methods rely on identification of the methylation state of these markers, which can be characterized using any suitable technique. Such techniques include, but are not limited to, sequencing (both pre- and post-bisulfite treatment), DNA amplification (e.g., PCR and related methods) using methylation-specific primers, amplification using primers that hybridize proximal (e.g., ‘upstream’) from a marker being characterized and probing using a methylation-specific probe (e.g., a probe sequence that includes a dye or fluorophore), amplification using primers that hybridize proximal (e.g., ‘upstream’) from a marker being characterized followed by hybridization to a methylation-specific probe sequence that is coupled to a solid phase, amplification using primers that hybridize proximal (e.g., ‘upstream’) from a marker being characterized followed by electrophoresis and transfer to a membrane, amplification using primers that hybridize proximal (e.g., ‘upstream’) from a marker being characterized followed by characterization of amplification products using mass spectrometry, etc.

Compositions of the inventive concept can include one or more primer sequences for use in DNA amplification reactions. Such primer sequences can be positioned proximal to a methylation marker site, and can be derived from sequence data based on the position of the methylation site within the genome (once that position is identified) and the known effects of bisulfite treatment using methods known in the art. In some embodiments a primer sequence can be a methylation-specific primer, and can be applied to a sample that has not been treated with bisulfite and/or post-bisulfite treatment. Such primer sequences can be provided as a primer pair that includes a primer complementary to the DNA strand complementary to the methylation marker site, such that repeated rounds of amplification (e.g., as in PCR) produce amplified DNA having a characteristic size. Compositions of the inventive concept can include one or more probe sequences that include a portion that is complementary to a methylation marker site. In some embodiments such probe sequences can include a dye, fluorophore, or other observable marker that provides a characteristic signal. In some embodiments two or more of such probe sequence can be provided that carry different observable markers, which can support multiplex assays. In some embodiments such probe sequences can be coupled to an insoluble support (e.g., a microarray, a microparticle, etc.). In such embodiments a number of probe sequences can be provided on solid supports that are encoded (e.g., by position within an array, by dye content and/or size of a microparticle, etc.) that permit identification of the associated probe sequence.

Embodiments of the inventive concept include components selected for identification of methylation status at specified sites in cfDNA (as discussed above) in addition to components utilized in immunoassay(s) directed to one or more tumor markers associated with EOC (e.g., CA125 and/or HE4). Such components include, but are not limited to, primary antibodies directed to said tumor markers, secondary antibodies directed to the primary antibodies, radiolabeled conjugates, enzyme conjugates, fluorescent conjugates, and/or luminescent conjugates.

Systems of the inventive concept can incorporate reagents that include compositions as described above as well as supporting reagents (e.g., buffers, enzymes, etc.). In some embodiments such systems can include liquid-handling equipment (pipettors, etc.) for measuring and dispensing reagents and/or test samples into testing receptacles. In some embodiments such systems can include subsystems for performing test reactions (e.g., thermal cyclers, hybridization incubators, washing subsystems, etc.) and/or analytical subsystems for detecting or characterizing signals obtained as a result of characterizing reactions (e.g., a colorimeter, spectrophotometer, fluorometer, particle size sorter, etc.). Such systems can include a controller that is in communication with such subsystems and controls their actions. Such a controller can include a database of testing protocols, and can record, store, and/or report testing results. In some embodiments such a controller can include an algorithm for analysis of test results and can report a probability of an individual having early and/or late stage EOC, and in some embodiments can report a prognosis for the individual. In some embodiments such an algorithm can incorporate or be implemented by an artificial intelligence (AI) algorithm.

### Identification of CfDNA Methylation Markers for Eoc Using a Sample Pooling Strategy

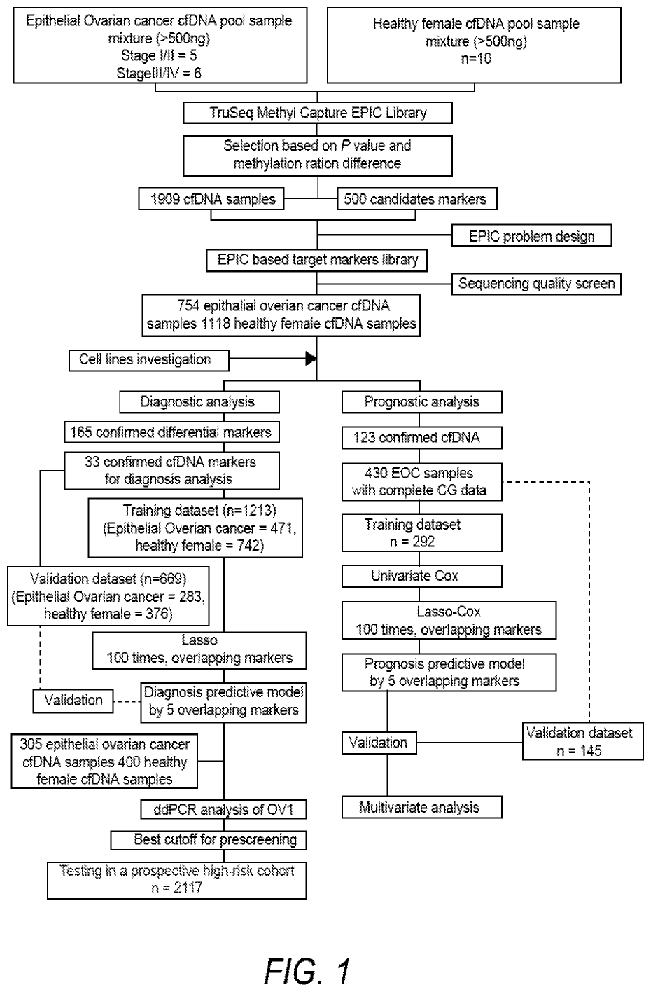

As is shown in FIG. 1, Inventors adapted a sensitive assay (TMC-EPIC® Kit) for the EOC methylation site marker discovery. A limitation of the current technology is the requirement of at least 500 ng DNA for library construction, which is very difficult to obtain from a single serum sample. To address this issue, Inventors constructed several libraries by using pooled cfDNA samples. 5 early stage pooled samples and 6 advanced stage pooled samples were gained from a total of >200-220 EOC patients, and the cfDNA of healthy subjects were derived from 10 healthy female pooled samples (total healthy subjects involved >200). These pool sample libraries are referred to as pool cohort in the following content (Table 1).

TABLE 1
Demographic characteristics of the three retrospective cohorts

| | Pool Cohort | | | Individual Cohort | | | ddPCR Cohort | | |
|---|---|---|---|---|---|---|---|---|---|
| | Healthy | Early | Advanced | Healthy | Early | Advanced | Healthy | Early | Advanced |
| | female | EOC | EOC | female | EOC | EOC | female | EOC | EOC |
| Total | 10 | 5 | 6 | 1118 | 205 | 549 | 480 | 111 | 194 |
| Age (years) | | | | | | | | | |
| Mean | | | | 45.5 | 46.7 | 52.8 | 44.2 | 49.6 | 53.6 |
| Median | | | | 45 | 49 | 53 | 44 | 48 | 53 |
| | | | | (20-88) | (13-80) | (9-84) | (22-80) | (21-78) | (22-76) |
| With CA125 | | | | 341 | 183 | 534 | 480 | 111 | 194 |
| data | | | | | | | | | |
| >35(U/mL) | | | | 13 | 81 | 270 | 24 | 43 | 1060 |
| <35(U/mL) | | | | 328 | 102 | 264 | 453 | 68 | 88 |
| With HE4 data | | | | | 183 | 532 | | | |
| >86.6(pmol/mL) | | | | | 13 | 46 | | | |
| <86.6(pmol/mL) | | | | | 170 | 486 | | | |

For each pooled sample, over 3.3 million CpG positions were examined, and our sequencing outcome gave on average 63.3 (21.7-126.2) reads at each CpG position. As a result, Inventors identified more than 268,039 differentially methylated loci (DML) or 21,104 differentially methylation regions (DMR) with a methylation value difference >10% when comparing EOC patients and healthy female cfDNA samples pools.

Validation of the Methylation Markers in Individual cfDNA Samples

As these markers were identified from the pool cohort, it is critical to verify them in individual samples to ensure that the representation in the pool samples were related to individuals. As plasma sample of each individual is limited, 500 CpG positions were selected based on their methylation difference and P-values between EOC and healthy pools as candidate markers for the individual validation. Accordingly, these markers' corresponding probes were designed, synthesized, and applied to the examination of 1909 individuals' cfDNA samples, all these samples are completely distinct from the samples constituting the pool cohort. The probes successfully captured 493 of the 500 candidate markers in these individual samples after UMI combination of the sequencing outcomes, 1872 samples (754 EOC patients and 1118 healthy female) gave in average more than 10 reads per CpG position, which were retained for the following analysis. These individual samples are referred to as individual cohort in following text (Table 1). The results revealed a good consistency of methylation change between individual and pool cohorts, and Inventors confirmed 165 markers with significant methylation difference (difference >10%, P<0.05).

Inventors also explored methylation status of the 500 CpG positions in seven ovarian tumor cell lines and an ovarian epithelial cell line. Positions exhibited over 10% methylation difference between the epithelial cell line and more than four of seven tumor cell lines were retained and overlapped with the 165 markers confirmed in individual samples. Consequently, 33 additional markers were identified as potential cfDNA markers.

### EOC Diagnostic Panel (OCDP) Construction

LASSO based selection: In some embodiments Inventors randomly divided the above examined individual cohort data into training dataset (471 EOC and 742 healthy female samples) and validation dataset (283 EOC and 376 healthy female samples). In the training dataset, Inventors analyzed the 33 ctDNA markers for separating samples of EOC patients from healthy female subjects by the least absolute shrinkage and selection operator (LASSO) and random forest. Markers identified in over 90 out of 100 times' LASSO were overlapped with the markers given by random forest analysis, the retained markers were applied to a logistic regression for diagnostic modeling. Based on this approach, eight markers were selected as potentially applicable to an EOC diagnostic panel (OCDP, Table 2). In some embodiments all eight markers are utilized as at least part of an EOC diagnostic panel. In some embodiments a portion (e.g., two or more) of these markers are utilized as at least part of an EOC diagnostic panel.

TABLE 2
Characteristics of eight methylation markers in
the OCDP and their coefficients in EOC diagnosis.

| Marker | | | | | | |
|---|---|---|---|---|---|---|
| ID | Position | Ref Gene | Coefficients | SE | z value | p value |
| | | | 15.065 | 1.1508 | 13.091 | <2e−16 |
| OV1 | chr2:12246596 | intergenic | −7.431 | 0.7973 | −9.321 | <2e−16 |
| OV2 | chr5:157030693 | intergenic | −3.2541 | 0.798 | −4.078 | 4.54e−05 |
| OV6 | chr5:34924294 | BRIX1 | −1.7539 | 0.5987 | −2.93 | 0.003395 |
| OV7 | chr6:36992585 | FGD2 | −3.877 | 0.8803 | −4.404 | 1.06E−05 |
| OV9 | chr2:205889942 | PARD3B | −1.9556 | 0.9317 | −2.099 | 0.035832 |
| OV11 | chr11:116852380 | SIK3 | 3.1249 | 0.6379 | 4.899 | 9.63E−07 |
| OV18 | chr6:30131220 | TRIM15 | −5.0339 | 0.9263 | −5.435 | 5.49E−08 |
| OV22 | chr10:369949 | DIP2C | −3.0527 | 0.8718 | −3.502 | 0.000462 |

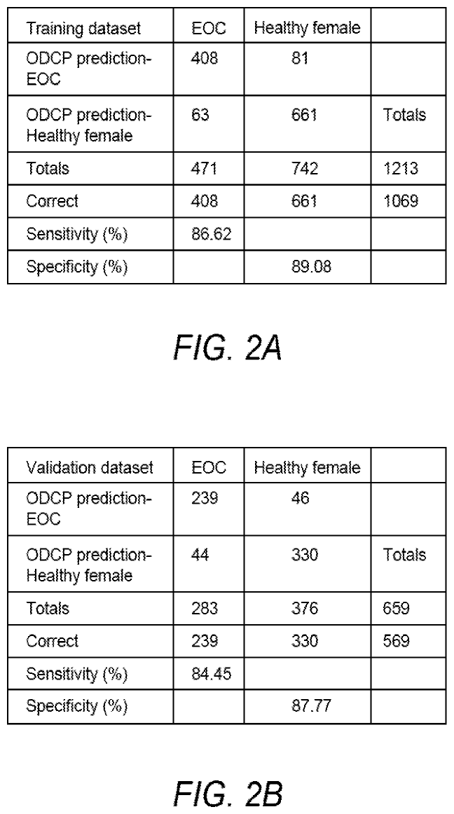

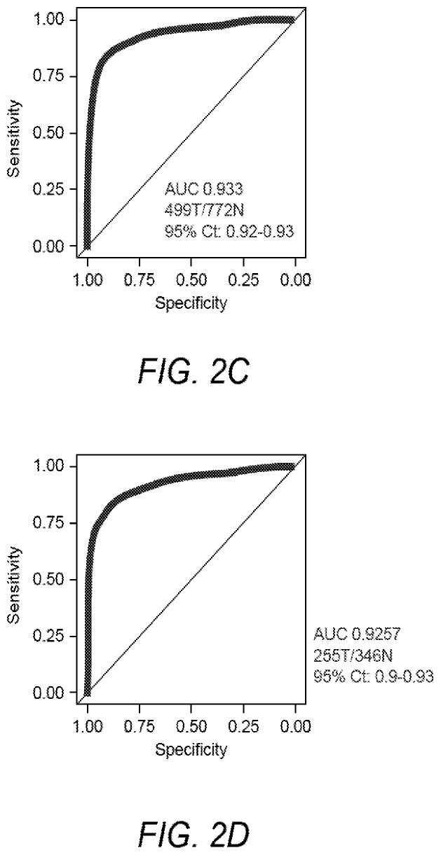

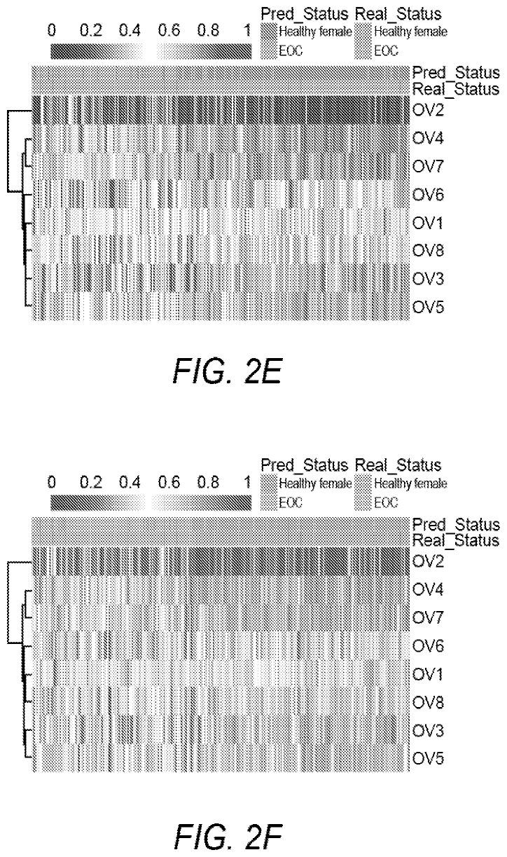

In some embodiments, a single marker from among the markers cited in Table 2 (e.g., OV1) can be used as at least part of an EOC diagnostic panel. Such diagnostic panels can be utilized in screening studies for EOC, staging of EOC, and/or to monitor treatment of EOC. Such diagnostic panels can incorporate additional cancer markers associated with EOC as are known in the art. Such diagnostic panels can be implemented using any suitable DNA detection and/or identification technology, such as DNA amplification, multiplexed DNA amplification, etc. In the training dataset, Inventors observed 86.62% sensitivity and 89.08% specificity (AUC=0.933) for EOC diagnosis by OCDP and when it was applied to the validation dataset, very similar sensitivity (84.45%) and specificity (87.77%) (AUC=0.9257) were also observed (FIGS. 2A to 2F). FIGS. 2A and 2B provide Confusion tables of binary results of the OCDP in the training and validation datasets, respectively. FIGS. 2C and 2D show typical ROC curves of the diagnostic prediction model with methylation markers in the training and validation data sets, respectively. FIGS. 2E and 2F show typical unsupervised hierarchical clustering of the seven methylation markers selected for use in the diagnostic prediction model in the training and validation data sets, respectively.

Furthermore, the positive predict value (PPV), negative predict value (NPV) and false positive rate (FPR) of the OCDP were 0.83, 0.91 and 0.11, respectively, in the training dataset, and were 0.84, 0.89 and 0.12, respectively, in the validation dataset. Because EOC and healthy individual datasets are not strictly age matched, to assess if our OCDP can be impacted by age, Inventors assessed cd-score of the OCDP in different age groups in the healthy female subject cohort and no significant variation among different age groups was observed.

Another important observation was that significant differences in methylation were also found at the CpG positions around these identified markers (differentially methylated regions, or DMRs), implying a functional correlation of these markers' methylation status with genetic expression. Indeed, in 6 DMRs that can be annotated to gene or gene regulatory region, most of their corresponding genes showed significant difference in expression between 426 ovarian cancer patients' and 88 healthy female's ovarian tissues.

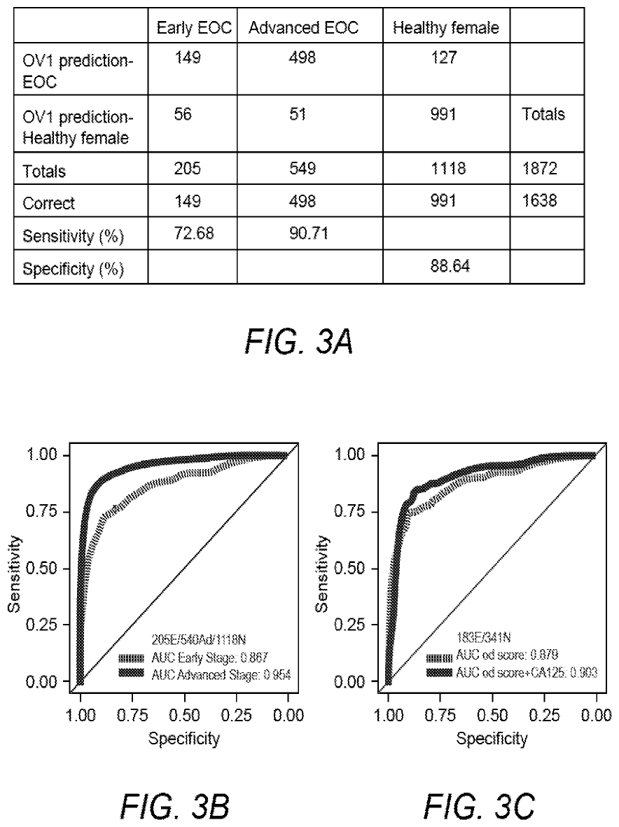

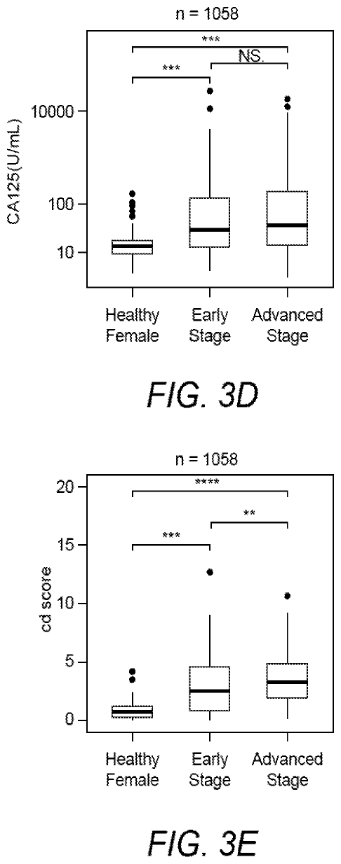

To determine the performance characteristics of an ovarian cancer diagnostic panel (OCDP) as shown in Table 2 with reference to disease stages, Inventors initially reviewed at early and advanced EOC separately in training and validation datasets, as a result, relatively consistent ROC curves of either stage was found between training and validation dataset. Results are shown in FIGS. 3A to 3E. FIGS. 3A to 3 show results of studies of the OCDP and CA125 for different stages of EOC. FIG. 3A provides Confusion tables of binary results of the diagnostic prediction model for different cancer stages of ovarian cancer and healthy female. FIG. 3B shows typical ROC curves of the OCDP for distinguishing early stage (red) or advanced stage (blue) EOC patients from healthy female. FIG. 3C shows typical ROC curves of the OCDP (red) and OCDP in combination with CA125 (blue) for distinguishing early stage EOC from healthy female. Note: only samples with CA125 information were summarized in this figure. FIG. 3D depicts CA12 levels of healthy female samples and EOC samples of different stages. FIG. 3E depicts CA12 OCDP cd-score values of healthy female samples and EOC samples of different stages.

Overall, in the individual cohort, OCDP as shown in Table 2 successfully identified 149 out of 205 early stage EOC (72.68% sensitivity) and 498 out of 549 advanced stage EOC (90.71% sensitivity), with an overall specificity of 88.64% (FIGS. 3A and 3B). In addition, Inventors estimated the corresponding sensitivities of OCDP when it was on different specificities (85%, 90%, 95%) in the individual cohort, and found that if Inventors adjust the OCDP specificity to >95%, its sensitivity would be still as high as over 75% (Table 3).

TABLE 3

| Model | Specificity: | 85.06% | 90.07% | 95.08% |
|---|---|---|---|---|
| OCDP | Early | 74.15% | 69.27% | 56.10% |
| | sensitivity | | | |
| | Advanced | 91.8% | 89.44% | 82.88% |
| | sensitivity | | | |
| | Overall EOC | 87.00% | 83.95% | 75.60% |
| | sensitivity | | | |

Corresponding sensitivities of OCDP when the specificities were over 85%, 90% and 95% in distinguishing EOC from healthy female.

When the OCDP as shown in Table 2 was used to assess for its diagnostic ability in EOC and endometriosis samples, not only the cd-score was significantly different between EOC samples of the individual cohort (n=754) and endometriosis sample of an independent cohort (n=96), but also an 85.81% sensitivity on 75% specificity was observed when OCDP was discriminating EOC from endometriosis samples.

Performance comparison between OCDP identified in LASSO analysis and conventional serum biomarkers: CA125 and HE4 assays are commonly used EOC screening tests in clinical practice despite their unsatisfactory sensitivities. When Inventors compared diagnostic performance of CA125 and HE4 with OCDP as shown in Table 2 in 715 samples of the individual cohort with complete CA125 and HE4 information, CA125 or HE4 alone provided a sensitivity of only 48.81% and 46.85%, respectively. When CA125 and HE4 were used in combination, sensitivity went up to 60.6%. In contrast, our OCDP demonstrated 86.71% sensitivity in these samples. Importantly, in the 282 EOC samples that were missed by CA125 and HE4 assay, 235 (83.33%) were correctly identified/diagnosed by the OCDP.

Inventors did not observe significant differences in CA125 biomarker levels between early and advanced stages EOC (FIG. 3D). However, using the OCDP Inventors observed a higher level of cd score for advanced stage when compared to the early stage EOC (FIG. 3E), suggesting a correlation with the tumor load.

Next, Inventors evaluated whether combination of the OCDP as shown in Table 2 with CA125 can improve sensitivity of the resulting combined test panel. In 1,058 samples of the individual cohort with CA125 information, combining our OCDP with CA125 enhanced the sensitivity by 6.14% (from 86.47% to 92.61%, n=717) but reduced specificity by 3.52% (from 90.62% to 87.1%, n=341) when compared with OCDP alone. Interestingly, for the early stage EOC patients (n=183), the sensitivity increased from 73.22% to 84.15% on 87.1% specificity in the combined model, more importantly, such sensitivity can be as high as nearly 80% or 70% when the specificity is over about 90% or 95% (FIG. 3C, Table 4 and 5).

TABLE 4
Specificities and sensitivities of the OCDP and CA125 for
distinguishing ovarian cancer from healthy female samples.

| | | CA125 | | OCDP | | OCDP + CA125 | |
|---|---|---|---|---|---|---|---|
| | Sample | Sensitivity | Specificity | Sensitivity | Specificity | Sensitivity | Specificity |
| | size | (%) | (%) | (%) | (%) | (%) | (%) |
| Healthy | 341 | | | | | | |
| female | | | | | | | |
| Early EOC | 183 | 44.26 | | 73.22 | | 84.15 | |
| Advanced | 534 | 50.26 | 96.19 | 91.01 | 90.62 | 95.51 | 87.10 |
| EOC | | | | | | | |
| Total EOC | 717 | 48.95 | | 86.47 | | 92.61 | |

Note:
only samples with CA125 information are summarized in this table.
Early: early stage EOC; Advanced: advanced stage EOC.

TABLE 5

| Model | Specificity: | 85.04% | 90.03% | 95.01% |
|---|---|---|---|---|
| OCDP + | Early | 84.70% | 79.78% | 69.04% |
| CA125 | sensitivity | | | |
| | Advanced | 95.69% | 94.38% | 89.51% |
| | sensitivity | | | |
| | Overall EOC | 92.88% | 90.66% | 84.38% |
| | sensitivity | | | |

Corresponding sensitivities of the OCDP + CA125 combined model when the specificities were over 85%, 90% and 95% in distinguishing EOC from healthy female.

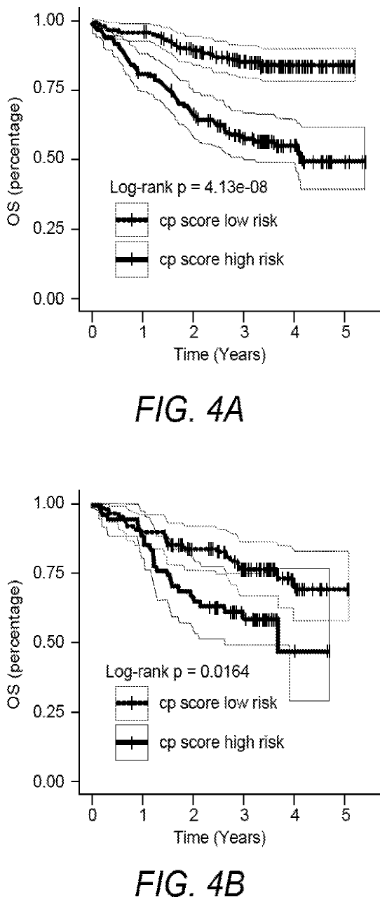

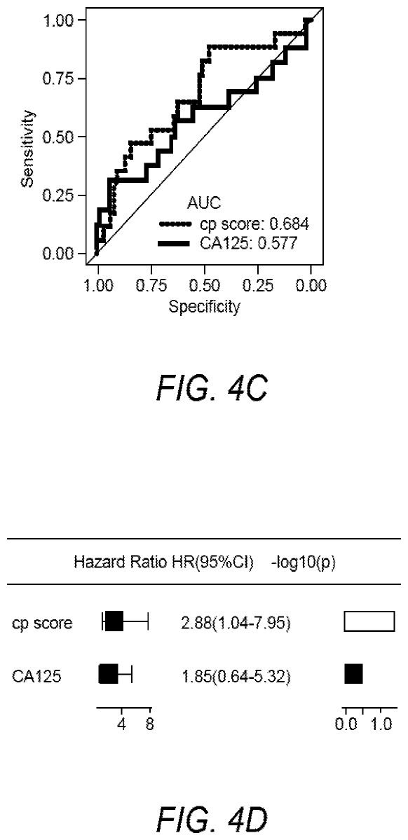

EOC prognostic prediction: To investigate the prognostic prediction potential of the 500 CpG positions selected from pool cohort, Inventors looked over their methylation status in the cell lines mentioned above. Results are shown in FIGS. 4A to 4D. FIG. 4A shows exemplary Kaplan-Meier plots for overall survival of EOC patients in the low- and high-risk groups determined by the OCDP as shown in Table 2 in the training datasets. FIG. 4B shows typical Kaplan-Meier plots for overall survival of EOC patients in the low- and high-risk groups determined by the OCPP in the validation dataset. FIG. 4C shows typical ROC curves and corresponding AUCs of 5-year survival prediction by OCPP cp score and CA125 and in early EOC. FIG. 4D shows results of multivariable analysis for early EOC survival with covariates including OCPP cp score and CA125.

124 markers were selected for prognostic analysis because they showed over 10% methylation change between EOC and healthy female in the individual cohort and such change was consistent in more than four out of seven ovarian tumor lines. 437 EOC patients in the individual cohort with complete survival information were selected and randomly split into training and validation datasets with 2:1 ratio. UniCox and LASSO were applied to reduce the dimensionality to three markers and a Cox-model of an EOC prognostic panel (OCPP) was constructed (Table 6). Kaplan-Meier curves were generated in training and validation datasets using a combined prognosis score (cp-score) of OCPP (FIGS. 4A and 4B). The high-risk group had 135 observations with 61 events in the training dataset, and 55 observations with 24 events in the validation dataset; while the low-risk group had 157 observations with 24 events in the training dataset, and 90 observations with 23 events in the validation dataset. The median survival time in the low-risk group was significantly shorter than that of the high-risk group by log-rank test in both the training (P<0.0001) and validation dataset (P=0.012) (FIGS. 4A and 4B).

TABLE 6
Characteristics of the three methylation markers
and their coefficients in EOC prognosis.

| Marker | | | | | | |
|---|---|---|---|---|---|---|
| ID | Position | Ref Gene | Coefficients | SE | z value | p value |
| | (Intercept) | | −1.163 | 1.073 | −1.083 | 0.2786 |
| OV15 | Chr5:118666498 | TNSFIP8 | 2.036 | 1.030 | 1.977 | 0.0480 |
| OV18 | chr6:30131220 | TRIM15 | −3.242 | 1.219 | −2.659 | 0.0078 |
| OV56 | Chr8:10135563 | RNF19A | 2.908 | 0.920 | 3.224 | 0.0013 |

In some embodiments, a single marker from among the markers cited in Table 6 can be used as at least part of an EOC diagnostic or prognostic panel. In some embodiments, two or more markers from among the markers cited in Table 6 can be used as at least part of an EOC diagnostic or prognostic panel. Such panels can incorporate additional cancer markers associated with EOC as are known in the art. Such diagnostic or prognostic panels can be implemented using any suitable DNA detection and/or identification technology, such as DNA amplification, multiplexed DNA amplification, etc.

Meanwhile, CA125 and cancer stage were also effective in EOC prognosis prediction, in aspect of AUC, HR and separation of KM curves, both factors exhibited similar power in EOC prognosis prediction compared with OPCC, and combining OPCC, CA125 and cancer stage dramatically improved prognosis predictive efficiency (Figure S10A-D, S11A-D). On the other hand, when Inventors looked at the early stage EOC (17 events in 126 observations), OPCC was still effective in their prognosis prediction, which AUC was 0.684 and HR was 2.88 (95% CI: 1.04-7.95), though CA125 showed reduced effectiveness in predicting prognosis of these observations.

EOC prognostic prediction using markers identified using LASSO and UniCox: Inventors also determined the prognostic prediction potential of the 493 methylation markers selected from the pool cohort. 151 markers were employed for prognostic analysis as showing over 10% methylation change between EOC and healthy females in the individual cohort. 437 EOC patients in the individual cohort with complete survival information were selected and randomly split into training and validation datasets with a 2:1 ratio. UniCox and LASSO were applied to reduce the dimensionality to three markers and a Cox-model of an EOC prognostic panel (OCPP) was constructed (FIG. 4A). Kaplan-Meier curves were generated in training and validation datasets using a combined prognosis score (cp-score) of OCPP (FIG. 4B and FIG. 4C). The high-risk group had 135 observations with 61 events in the training dataset, and 55 observations with 24 events in the validation dataset. The low-risk group had 157 observations with 24 events in the training dataset, and 90 observations with 23 events in the validation dataset. The median survival time in the high-risk group was significantly shorter than that of the low-risk group by log-rank test in both the training (P<0.0001) and validation dataset (P=0.012) (FIG. 4B and FIG. 4C).

### MethylBERT Based Selection

LASSO based dimensionality reduction followed by logistic regression for binary classification is a classic diagnostic model construction strategy, but has certain limitations. Such an approach is limited by the number of biomarkers that can be included for modeling due to constraints on Events Per Variable (EPV), a ratio between feature numbers and sample size. For example, a prediction model built on a logistic regression analysis generally adopts >10 EPV for a good perdition. Accordingly, no more than 75 methylation markers could be selected in a study for logistic regression modeling with a sample size of 754 (as in EOC cohorts described herein). Moreover, in sequencing cfDNA samples extracted from 2 mL plasma to saturation, less than 35% DNA bases of a whole genome were found to be over 10× in depth. This implies that a large number of methylation markers may not be exposed by analyzing cfDNA sequencing data in this fashion. Accordingly, a method that was not limited by the number of input markers and was also able to simulate unexamined from examined methylation sites would be more suitable for cfDNA methylation marker dependent diagnostic modeling.

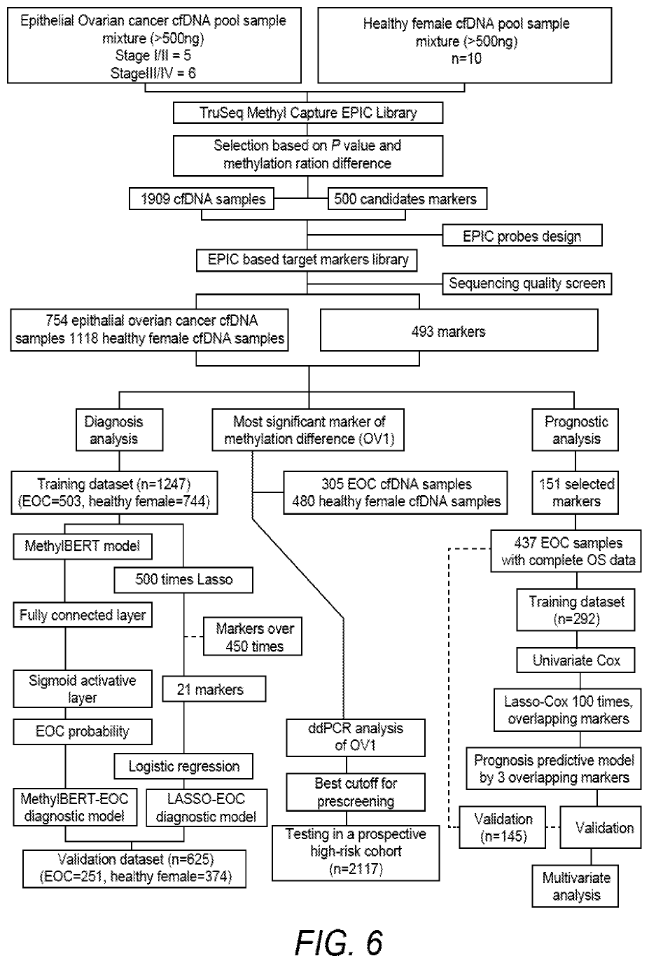

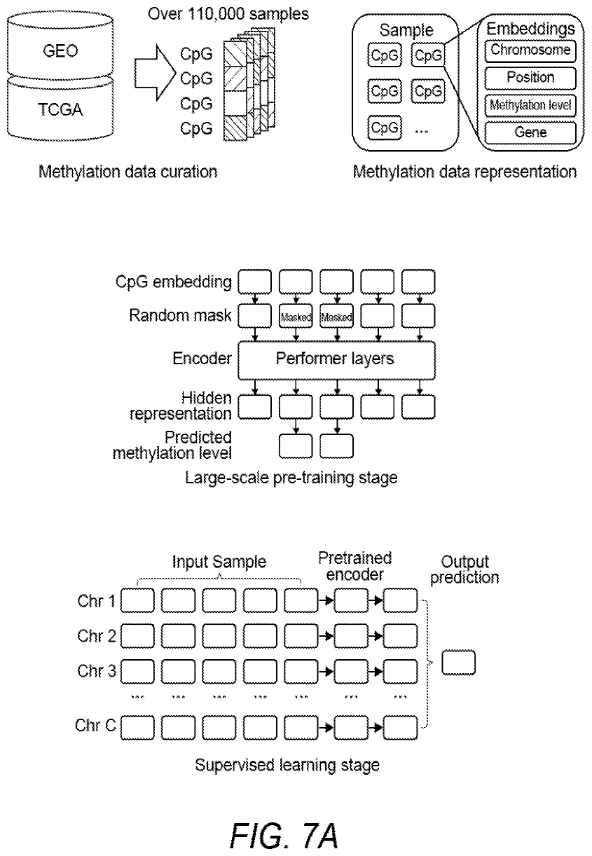

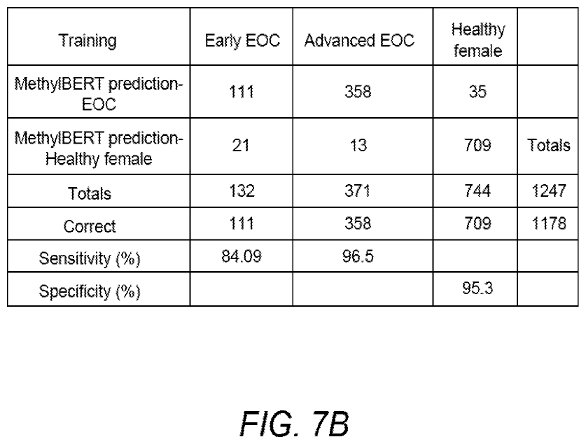

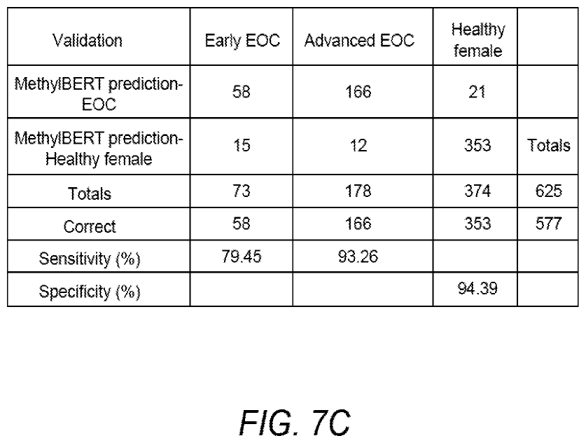

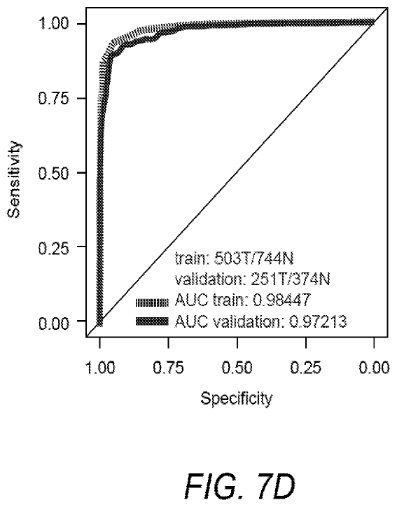

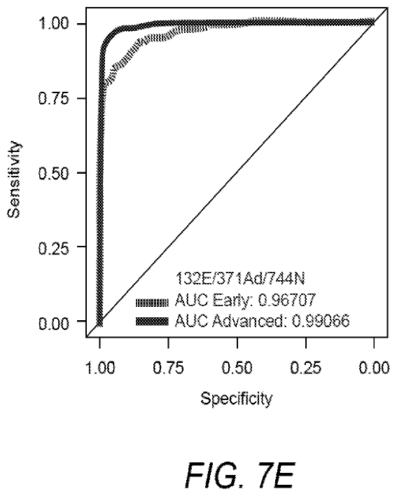

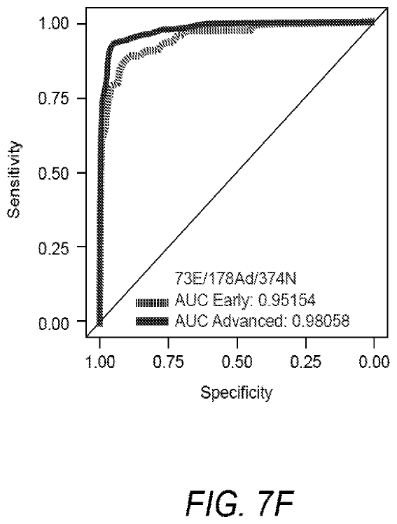

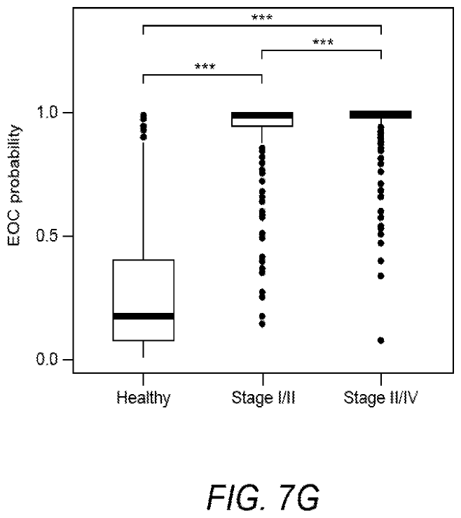

Inventors have developed an approach in methylation marker discovery using a deep learning language model, which can overcome the above-mentioned limitations of the conventional LASSO approach. An overview of the derivation this model is provided in FIG. 6. Inventors employed bidirectional encoder representations from transformers (BERT), a Transformer-based language prestation model that can learn broad clinical and biological knowledge and feature representations, and applied the BERT paradigm to analyze all available cancer DNA methylation datasets to exploit massive knowledge and interactions among chromosome, position, methylation level and gene function in over 90,000 cancer samples from GEO and TCGA. Inventors then constructed a model that enabled a system to learn individual methylation CpG site representations and multiple CpG-CpG site relationships. This was designated MethylBERT (FIG. 7A). Thereafter, MethylBERT was applied to analyze a training dataset that was randomly selected from ⅔ of the individual cohort, binary classification using fully connected layer and sigmoid activation function conferred an EOC probability to each sample, hence a MethylBERT-EOC diagnostic model was built. FIGS. 7A to 7D depict aspects of the MethylBERT model, analysis using MethylBERT, and typical results.

FIG. 7A provides a schematic overview of the MethylBERT model. Over 110,000 WGBS and RRBS data were collected from GEO and TCGA datasets (upper left), their chromosome embedding, position embedding, methylation level embedding and gene embedding were combined as CpG site embedding scheme (upper right), which was fed into a matrix decomposition-based Transformer model with a certain percentage of CpG sites randomly masked, and pretrained it to predict the methylation level of the masked CpG sites, with the given context of their surrounding CpG sites (lower left). The fine-tuning pretrained model (MethylBERT) was employed to process the methylation data of input samples (lower right). FIGS. B and C provide Confusion tables of binary results of MethylBERT-EOC diagnostic model in the training and validation datasets, respectively. FIG. 7D depicts typical ROC curves of MethylBERT-EOC diagnostic model in EOC diagnostic prediction the training and validation data sets. FIGS. 7E and 7F provide typical ROC curves of MethylBERT-EOC diagnostic model results in early and advanced EOC diagnostic prediction the training and validation datasets, respectively. FIG. 7G shows typical MethylBERT-EOC diagnosis based EOC prediction score of healthy female samples and EOC samples of different stages. (**P<0.01, ***P<0.001, ****P<0.0001; NS, not significant).

MethylBERT-EOC diagnostic model gave 93.24% sensitivity on 95.3% specificity (AUC=0.98) in a training dataset that consisted of 503 EOC and 744 healthy female samples and 89.24% sensitivity on 94.39% specificity (AUC=0.97) in the validation dataset of 251 EOC and 374 healthy female samples (FIGS. 7B to 7D). The positive predictive value (PPV), negative predictive value (NPV), and false positive rate (FPR) were 93.06%, 95.3%, and 4.71%, respectively, in the training dataset, and were 91.43%, 94.39%, and 5.53%, respectively, in the validation dataset. Furthermore, in the training dataset, there were 132 early EOC samples, MethylBERT-EOC diagnostic model could successfully diagnose 111 of them (84.09% sensitivity), while in the validation dataset of 73 early EOC samples, the model diagnosed 58 of them (79.45% sensitivity) (FIGS. 7C, 7E, and 7F).

Since EOC and healthy subjects in the individual cohort were not strictly age matched MethylBERT-EOC diagnostic model was assessed for potential impact by age difference. No significant variation among different age groups was observed. In addition, the corresponding estimated sensitivities of MethylBERT-EOC diagnostic model at different specificities (85%, 90%, 95%, 99%) showed that at a specificity at >99% sensitivity would be similar to that estimated at a sensitivity of over 70% in the validation dataset (Table 7).

TABLE 7

| Model | Specificity: | 85.03% | 90.10% | 95.19% | 99.20% |
|---|---|---|---|---|---|
| OCDP | Early | 90.41% | 87.67% | 79.45% | 63.01% |
| | sensitivity | | | | |
| | Advanced | 96.07% | 94.38% | 93.26% | 74.16% |
| | sensitivity | | | | |
| | Overall EOC | 94.42% | 92.43% | 89.24% | 89.24% |
| | sensitivity | | | | |

To determine if MethylBERT-EOC diagnostic model was able to discriminate EOC from other gynecological disease, Inventors examined the 493 markers in 96 endometriosis cfDNA samples and applied their methylation data to the model. Not only the EOC probability was significantly lower in these endometriosis samples compared to EOC samples of the validation dataset, but also an 89.24% sensitivity on 91.66% specificity was observed to discriminate the EOC samples from endometriosis samples.

Inventors believe that the MethylBERT artificial intelligence approach can be used in an EOC diagnostic model to accurately and specifically identify cfDNA samples obtained from individual with EOC, monitor treatment of EOC in individuals with the disease, and/or aid in determining prognosis of individuals with EOC. Inventors further believe that the MethylBERT artificial intelligence approach is generally applicable to disease states believed to be associated with methylation of specific sites within the genome. The MethylBERT artificial intelligence algorithm permits analysis of larger datasets than previous approaches, providing increased sensitivity, increased sensitivity, and/or reduced time required for analysis and/or time required for identification of affected individual on a given computational platform relative to current DNA diagnostic approaches.

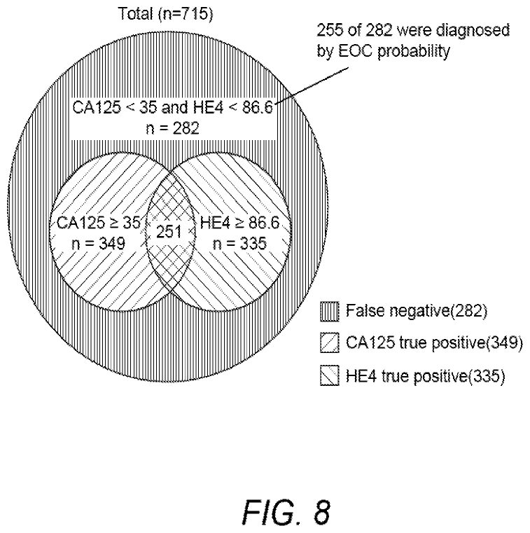

Performance comparison between MethylBERT analysis and conventional serum biomarkers: As noted above, CA125 and HE4 assays are commonly used EOC screening tests in clinical practice, despite their unsatisfactory sensitivities. Inventors compared diagnostic sensitivities of CA125 and HE4 with a MethylBERT-EOC diagnostic model (as shown in Table 10) using 715 EOC samples of an individual cohort having complete CA125 and HE4 information. CA125 or HE4 alone provided a sensitivity of 48.81% and 46.85%, respectively, for these samples. When CA125 and HE4 were used in combination, the sensitivity increased to 60.6%. In contrast, MethylBERT-EOC diagnostic model demonstrated 92.73% sensitivity in these samples. Importantly, of the 282 EOC samples that were missed by CA125 and HE4 assay, 255 (90.43%) were correctly diagnosed by MethylBERT-EOC diagnostic model (FIG. 8).

Inventors did not observe a significant difference between early and advanced stage EOC in the individual cohort (n=1058 that possessed CA125 information). However, applying MethylBERT-EOC diagnostic model to the same EOC samples showed higher EOC probability in advanced EOC (FIG. 7G), suggesting a potential correlation with the tumor load. This suggests that MethylBERT-EOC diagnostic model can be useful in tracking disease progression and treatment efficacy monitoring, as well as having a role in monitoring disease recurrence.

Inventors also evaluated the effect of combining MethylBERT-EOC diagnostic model with CA125 on sensitivity of EOC detection. In 1,058 samples of an individual cohort with CA125 information, combining MethylBERT-EOC diagnostic model with CA125 detection enhanced the sensitivity by 3.21% (from 92.47% to 95.68%) but reduced specificity by 3.81% (from 97.36% to 93.55%) when compared with MethylBERT-EOC diagnostic model alone. However, for early stage EOC samples (n=183), the sensitivity increased dramatically from 82.51% to 89.62% at 93.55% specificity in the combined model. Notably, such sensitivity can be as high as about 85% when the specificity is over about 95% (Table 8 and 4).

TABLE 8

| | | CA125 | | MethylBERT-EOC model | | Combined | |
|---|---|---|---|---|---|---|---|
| | | Sensitivity | Specificity | Sensitivity | Specificity | Sensitivity | Specificity |
| | Sample size | (%) | (%) | (%) | (%) | (%) | (%) |
| Healthy female | 341 | | | | | | |
| Early EOC | 183 | 44.26 | | 82.51 | | 89.62 | |
| Advanced EOC | 534 | 50.26 | 96.19 | 95.88 | 97.36 | 97.75 | 93.55 |
| Total EOC | 717 | 48.95 | | 92.47 | | 95.68 | |

TABLE 9

| Model | Specificity: | 85.04% | 90.07%% | 95.01%% |
|---|---|---|---|---|
| OCDP | Early | 93.44% | 90.16% | 85.79% |
| | sensitivity | | | |
| | Advanced | 98.31% | 97.38% | 96.44% |
| | sensitivity | | | |
| | Overall EOC | 97.07% | 95.54% | 93.72% |
| | sensitivity | | | |

Comparison between MethylBERT-EOC diagnostic model and the LASSO-logistic regression EOC diagnostic model: Using the same training dataset of MethylBERT-EOC diagnostic modeling, Inventors developed a diagnostic model by using conventional LASSO-logistic regression strategy as described above. To reduce the number of markers, the 493 markers were analyzed by 500 times LASSO. The 21 markers identified as over 450 times LASSO were subsequently applied to logistic regression for diagnostic modeling. In this way, a LASSO-logistic regression EOC diagnostic model based on the same training set as MethylBERT EOC diagnostic model was obtained (Table 10).

TABLE 10

| Marker Alias | Position | Ref Gene | Coefficients | SE | z value | P value |
|---|---|---|---|---|---|---|
| | | | 12.7533 | 2.5782 | 4.947 | 7.55E−07 |
| OV1 | chr2:12246596 | intergenic | −8.3196 | 0.9763 | −8.522 | 2.00E−16 |
| OV2 | chr21:36421468 | RUNX1 | 6.7281 | 1.4829 | 4.537 | 5.70E−06 |
| OV3 | chr15:101783038 | CHSY1 | −6.3064 | 1.754 | −3.595 | 0.000324 |
| OV4 | chr8:124195403 | FAM83A | −5.9298 | 1.8072 | −3.281 | 0.001033 |
| OV5 | chr1:55393910 | intergenic | 4.7989 | 1.8548 | 2.587 | 0.009674 |
| OV6 | chr2:205889911 | PARD3B | −4.4206 | 1.3043 | −3.389 | 0.000701 |
| OV7 | chr5:88036833 | LINC00461 | 4.3654 | 0.9205 | 4.742 | 2.11E−06 |
| OV8 | chr17:26941529 | RSKR | 3.8698 | 1.2623 | 3.066 | 0.002171 |
| OV9 | chr8:144300733 | intergenic | −3.8243 | 1.3001 | −2.941 | 0.003266 |
| OV10 | chr10:369949 | DIP2C | −3.8022 | 1.1517 | −3.301 | 0.000962 |
| OV11 | chr1:109189642 | intergenic | 3.5558 | 1.2359 | 2.877 | 0.004015 |
| OV12 | chrX:117480838 | WDR44 | 3.5209 | 1.0351 | 3.401 | 0.00067 |
| OV13 | chr5:157030693 | intergenic | −3.4929 | 1.0378 | −3.366 | 0.000763 |
| OV14 | chr13:49323975 | intergenic | −3.3885 | 0.9786 | −3.462 | 0.000535 |
| OV15 | chr1:45250302 | BEST4 | 3.2178 | 1.4191 | 2.267 | 0.02336 |
| OV16 | chr6:30131220 | TRIM15 | −2.4697 | 1.1542 | −2.14 | 0.032381 |
| OV17 | chr2:158900312 | UPP2 | 1.5957 | 0.8696 | 1.835 | 0.066517 |
| OV18 | chr5:34924294 | BRIX1 | −1.5759 | 0.7785 | −2.024 | 0.042931 |
| OV19 | chr17:73631587 | SMIM5 | 1.5519 | 1.0131 | 1.532 | 0.12557 |
| OV20 | chr15:52436335 | GNB5 | 0.8596 | 0.6583 | 1.306 | 0.191649 |
| OV21 | chr10:45948653 | MARCHF8 | −0.3651 | 1.0123 | −0.361 | 0.718381 |

In some embodiments, a single marker from among the markers cited in Table 10 (e.g., OV1) can be used as at least part of an EOC diagnostic panel. Such diagnostic panels can be utilized in screening studies for EOC, staging of EOC, and/or to monitor treatment of EOC. Such diagnostic panels can incorporate additional cancer markers associated with EOC as are known in the art. Such diagnostic panels can be implemented using any suitable DNA detection and/or identification technology, such as DNA amplification, multiplexed DNA amplification, etc. Exemplary primer and probe sequences suitable for amplification and identification of sites noted in Table 10 are provided in Table 11. The Applicant notes that sequences for suitable amplification primer (e.g., forward and/or reverse) and probe sequences can be derived from the specific methylation site locations cited in Table 10 and available human genome sequence data using conventional methods and tools.

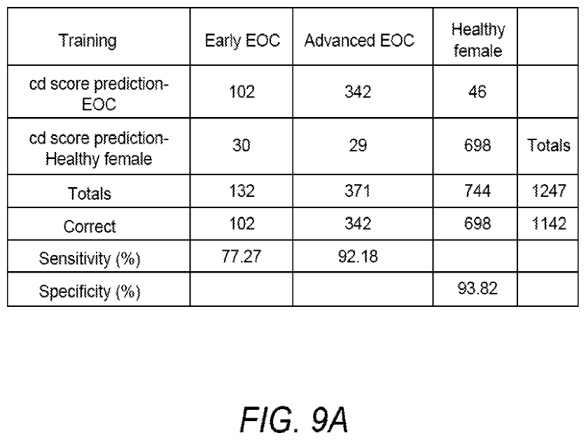

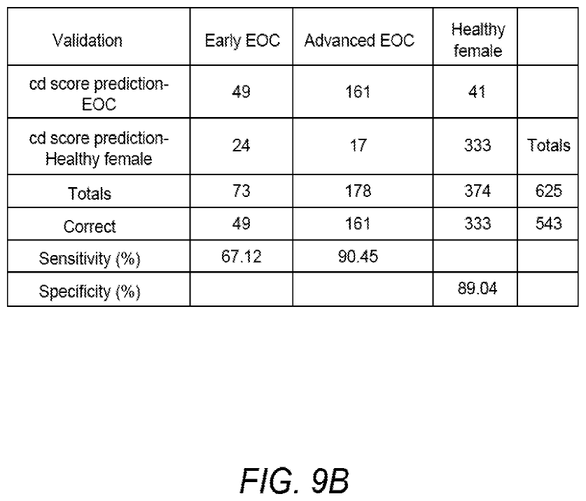

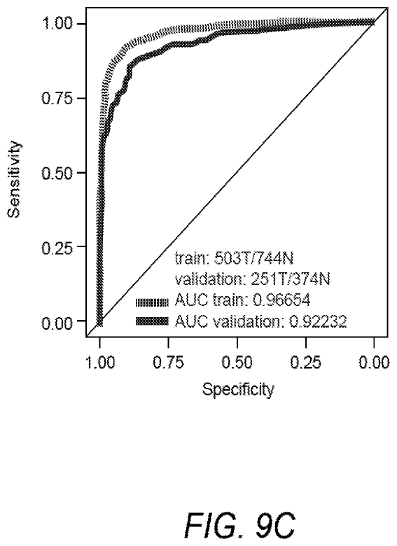

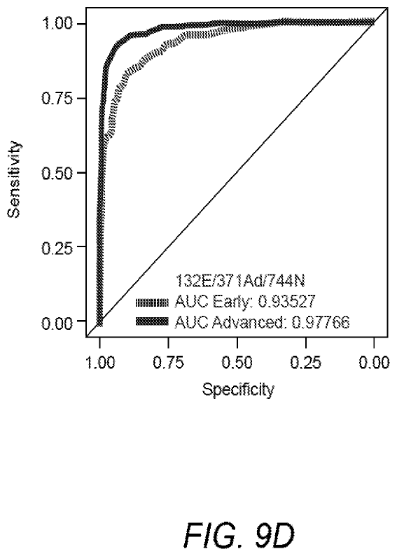

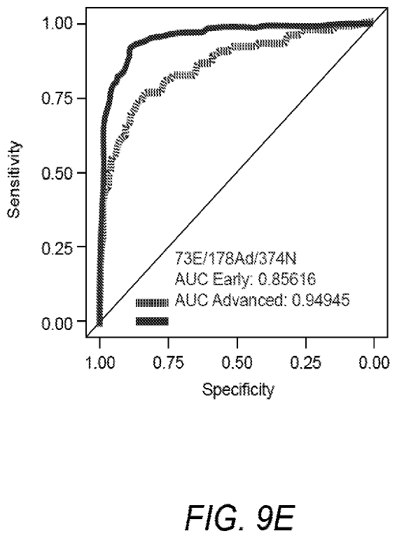

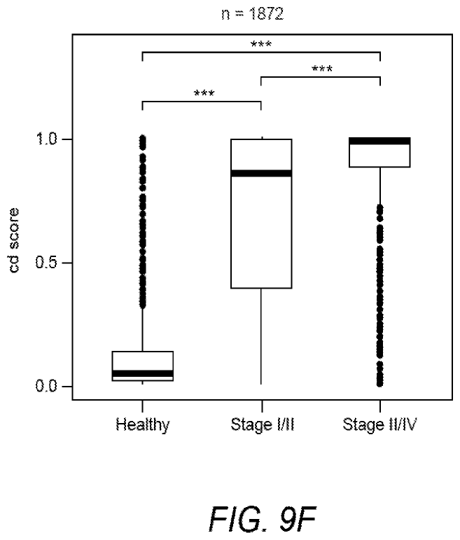

Results of application of this LASSO logistic regression model are provided in FIGS. 9A to 9F. FIG. 9A provides a Confusion table of binary results of the LASSO-EOC diagnostic model in the training dataset. FIG. 9B provides a Confusion table of binary results of the LASSO-EOC diagnostic model in the validation dataset. FIG. 9C shows typical ROC curves of the LASSO-EOC diagnostic model in EOC diagnostic prediction the training and validation data sets. FIG. 9D shows typical ROC curves of the LASSO-based EOC diagnostic model in early and advanced EOC diagnostic prediction the training dataset. FIG. 9E shows typical ROC curves of the LASSO-based EOC diagnostic model in early and advanced EOC diagnostic prediction in the validation dataset. FIG. 9F provides results from LASSO-EOC diagnosis based EOC prediction score of healthy female samples and EOC samples of different stages.

In the training dataset, this LASSO-logistic regression EOC diagnostic model exhibited 88.27% sensitivity on 93.82% specificity (AUC=0.97) for EOC diagnosis. When applied to the same validation dataset as used with MethylBERT-EOC diagnostic model, the LASSO-logistic regression EOC diagnostic model gave 83.67% sensitivity at 89.04% specificity (AUC=0.92) (FIGS. 9A to 9C). Both of these were lower than those given by MethylBERT-EOC diagnostic model. In addition, this LASSO-logistic regression EOC diagnostic model also showed significant difference in the combined diagnosis score (cd-score) between early and advanced stage EOC samples, however, it only identified 49 out of 73 early EOC, gave a 67.12% sensitivity, in the validation dataset (FIG. 9E), which was 12% lower than that was given by MethylBERT-EOC diagnostic model.

Development of a Simple and Cost-Effective ddPCR Assay

As noted above, Inventors believe that the MethylBERT artificial intelligence approach can be used to identify individuals with EOC and/or assist in providing a prognosis for individuals with EOC, using cfDNA obtained from blood samples. In some circumstances, however, resources may not be available to support this approach. Accordingly, with the good performance characteristics of the MethylBERT-derived OCDP in mind, Inventors evaluated the potential of developing a simple and cost-effective PCR based assay directed to diagnosis and/or prognosis of EOC. Inventors chose the most statistically significant methylation marker, OV1, to design a ddPCR assay and validated its utility in an independent cohort (referred to as ddPCR cohort, Table 1) of 305 EOC patients and 480 healthy female subjects. As noted above, suitable primer and probe sequences can be derived from known sequence data and the identification of the position of the OV1 methylation marker shown above. Sequences of suitable methylation and post-bisulfite treatment prove sequences and forward and reverse primers as used in this exemplary ddPCR assay are shown in Table 11, which also provides exemplary primer and probe sequences for amplification of methylation sites associated with EOC as identified herein.

TABLE 11

| Function | Sequence ID | Sequence |
|---|---|---|
| OV1 Methylated probe | SEQ ID NO. 1 | GGAAGGACGGTTTTTTG |
| OV1 Non-methylated probe | SEQ ID NO. 2 | GGAAGGATGGTTTTTTGG |
| OV1 Forward primer | SEQ ID NO. 3 | ATGATTTATAGGGTTGG |
| OV1 Reverse primer | SEQ ID NO. 4 | AACTTCCCCAACAC |
| OV2 Methylated probe | SEQ ID NO. 5 | CGCAGCGTGGTGAGC |
| OV2 Non-methylated probe | SEQ ID NO. 6 | CGCAGCATGGTGAGC |
| OV2 Forward primer | SEQ ID NO. 7 | GGCTGTGGGTTGGTGAT |
| OV2 Reverse primer | SEQ ID NO. 8 | CATAGTGGTCAGCAGGCAG |
| OV3 Methylated probe | SEQ ID NO. 9 | CCACATACCCGGAAACCAC |
| OV3 Non-methylated probe | SEQ ID NO. 10 | CCACATACCCAGAAACCAC |
| OV3 Forward primer | SEQ ID NO. 11 | CCCTCATCTCCATCTTCTGC |
| OV3 Reverse primer | SEQ ID NO. 12 | GCACIATGAGACAGTTAAAGG |
| OV4 Methylated probe | SEQ ID NO. 13 | AGGTGCCGGACTGTAGG |
| OV4 Non-methylated probe | SEQ ID NO. 14 | AGGTGCCAGACTGTAGG |
| OV4 Forward primer | SEQ ID NO. 15 | CIGAAGCAGGCCCTAAG |
| OV4 Reverse primer | SEQ ID NO. 16 | CTTCAGGTAGGGCTTCTCAG |
| OV5 Methylated probe | SEQ ID NO. 17 | TGAGCCCGGCATGTTGA |
| OV5 Non-methylated probe | SEQ ID NO. 18 | TGAGCCCAGCATGTTGA |
| OV5 Forward primer | SEQ ID NO. 19 | GCTAGAATGCAGTGACIC |
| OV5 Reverse primer | SEQ ID NO. 20 | CACAGTCICTCACCTCTG |
| OV6 Methylated probe | SEQ ID NO. 21 | TCTAAGTGCCGCAGACTGAA |
| OV6 Non-methylated probe | SEQ ID NO. 22 | TCTAAGTGCCACAGACTGAA |
| OV6 Forward primer | SEQ ID NO. 23 | AATATTGAGCTTCCCCAGGC |
| OV6 Reverse primer | SEQ ID NO. 24 | ACTCCTGACTAATAGCCITG |
| OV7 Methylated probe | SEQ ID NO. 25 | TGATGTGTTCGCAGCAGT |
| OV7 Non-methylated probe | SEQ ID NO. 26 | TGATGTGTTCACAGCAGT |
| OV7 Forward primer | SEQ ID NO. 27 | GAGAGAGCATGACAGAAGAGA |
| OV7 Reverse primer | SEQ ID NO. 28 | TCACTTTGAACITTATGAACTTC |
| OV8 Methylated probe | SEQ ID NO. 29 | CCCTTCCCCGTAGGATAT |
| OV8 Non-methylated probe | SEQ ID NO. 30 | CCCTTCCCCATAGGATAT |
| OV8 Forward primer | SEQ ID NO. 31 | CATGCACTCAACACACACAC |
| OV8 Reverse primer | SEQ ID NO. 32 | GGGAGGATGGCAGTAGGA |
| OV9 Methylated probe | SEQ ID NO. 33 | TTGAGCTCGAGCCTCATG |
| OV9 Non-methylated probe | SEQ ID NO. 34 | TTGAGCTCAAGCCTCATG |
| OV9 Forward primer | SEQ ID NO. 35 | GGTATCITGCTTAGCTGTCT |
| OV9 Reverse primer | SEQ ID NO. 36 | ATCCTGGATCTGCACCI |
| OV10 Methylated probe | SEQ ID NO. 37 | CAGCCGTAAGTGCAGAG |
| OV10 Non-methylated probe | SEQ ID NO. 38 | CAGCCATAAGTGCAGAG |
| OV10 Forward primer | SEQ ID NO. 39 | CACTCTGCTTGTTTCTATCACC |
| OV10 Reverse primer | SEQ ID NO. 40 | CAGTGAAAGGGCIGAGTC |
| OV11 Methylated probe | SEQ ID NO. 41 | GTTACGCAACACGACTG |
| OV11 Non-methylated probe | SEQ ID NO. 42 | GTTACACAACACGACTG |
| OV11 Forward primer | SEQ ID NO. 43 | GCCTGAAGACCACATTCTGA |
| OV11 Reverse primer | SEQ ID NO. 44 | ACACACAAGCTGTCTGGTAA |
| OV12 Methylated probe | SEQ ID NO. 45 | CAGGCACGTAAGCTGTTC |
| OV12 Non-methylated probe | SEQ ID NO. 46 | CAGGCACATAAGCTGTTC |
| OV12 Forward primer | SEQ ID NO. 47 | CCCITTTCTCTTCTGATCCA |
| OV12 Reverse primer | SEQ ID NO. 48 | AGCTCAGAACCCCACAGATA |
| OV13 Methylated probe | SEQ ID NO. 49 | ACCTTGTGCGTTTGTTGC |
| OV13 Non-methylated probe | SEQ ID NO. 50 | ACCTTGTGCATTTGTTGC |
| OV13 Forward primer | SEQ ID NO. 51 | AAGGGTTTTCTTTGAAGGCC |
| OV13 Reverse primer | SEQ ID NO. 52 | CTGTGCCTGTGTCATTTACTAT |
| OV14 Methylated probe | SEQ ID NO. 53 | AACAGTTCGAATGCGCC |
| OV14 Non-methylated probe | SEQ ID NO. 54 | AACAGTTCAAATGCGCC |
| OV14 Forward primer | SEQ ID NO. 55 | CACAGCATCAGAGGGTCTC |
| OV14 Reverse primer | SEQ ID NO. 56 | TCAAAGGTCAGTTCICTCTT |
| OV15 Methylated probe | SEQ ID NO. 57 | CAACCTGCGGTGAGTGG |
| OV15 Non-methylated probe | SEQ ID NO. 58 | CAACCTGCAGTGAGTGG |
| OV15 Forward primer | SEQ ID NO. 59 | AGAGGCTGAGCIAGGAC |
| OV15 Reverse primer | SEQ ID NO. 60 | CTCATTCCTGGGCTCCAC |
| OV16 Methylated probe | SEQ ID NO. 61 | CCAGGGCGTCTCGAAT |
| OV16 Non-methylated probe | SEQ ID NO. 62 | CCAGGGCATCTCGAAT |
| OV16 Forward primer | SEQ ID NO. 63 | AAAGAACTGGCTTACTTGGC |
| OV16 Reverse primer | SEQ ID NO. 64 | GCCTCTTGTGTTCCCTAGAA |
| OV17 Methylated probe | SEQ ID NO. 65 | CAAACCGCCCCCAGAG |
| OV17 Non-methylated probe | SEQ ID NO. 66 | CAAACCACCCCCAGAG |
| OV17 Forward primer | SEQ ID NO. 67 | GATCTTGCCCATATTGCTGC |
| OV17 Reverse primer | SEQ ID NO. 68 | CAGATTCCAGAGACTAAACTTGT |
| OV18 Methylated probe | SEQ ID NO. 69 | CACTTATCGTGCCAAACTC |
| OV18 Non-methylated probe | SEQ ID NO. 70 | CACTTATCATGCCAAACTC |
| OV18 Forward primer | SEQ ID NO. 71 | CATTCAATGGCAGCIGTTAG |
| OV18 Reverse primer | SEQ ID NO. 72 | TTGCTGTTCTCTTCCACCTC |
| OV19 Methylated probe | SEQ ID NO. 73 | CCAGGGCGGGAGGAAG |
| OV19 Non-methylated probe | SEQ ID NO. 74 | CCAGGGCAGGAGGAAG |
| OV19 Forward primer | SEQ ID NO. 75 | TCTAACACCAGCAGCAGAAA |
| OV19 Reverse primer | SEQ ID NO. 76 | TGGAAGTAGAAAGTGGTGCT |
| OV20 Methylated probe | SEQ ID NO. 77 | CCAGGCCCGGATAGATAG |
| OV20 Non-methylated probe | SEQ ID NO. 78 | CCAGGCCCAGATAGATAG |
| OV20 Forward primer | SEQ ID NO. 79 | CCATCCCTCCACTCTGAATT |
| OV20 Reverse primer | SEQ ID NO. 80 | TCACTAAATGGAATGCAGCI |
| OV21 Methylated probe | SEQ ID NO. 81 | CTTGGCCTCGAAAAAGCC |
| OV21 Non-methylated probe | SEQ ID NO. 82 | CTTGGCCTCAAAAAAGCC |
| OV21 Forward primer | SEQ ID NO. 83 | GTCATCTGGTTGAGCTCIA |
| OV21 Reverse primer | SEQ ID NO. 84 | CACTGTGACATGCAATCCTG |

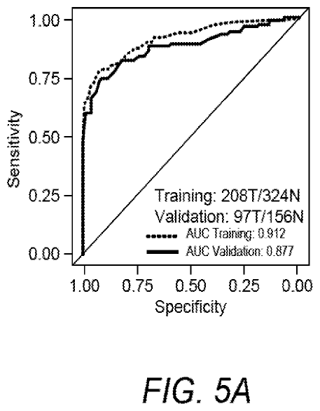

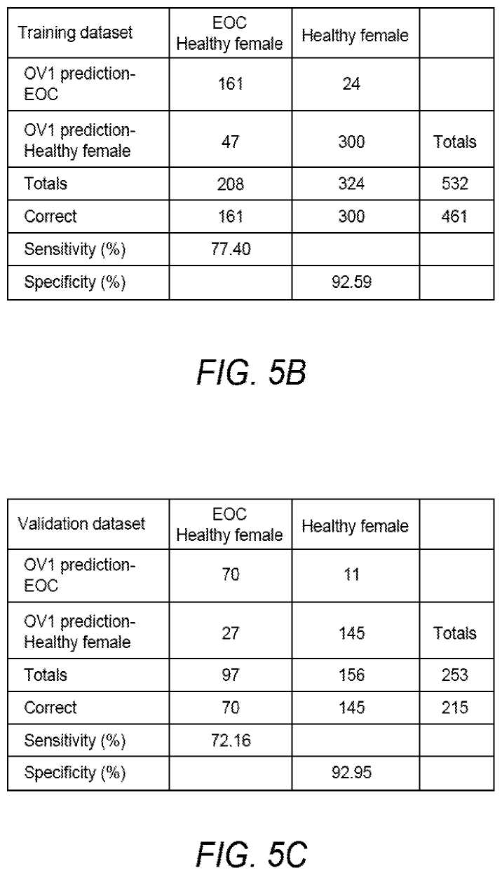

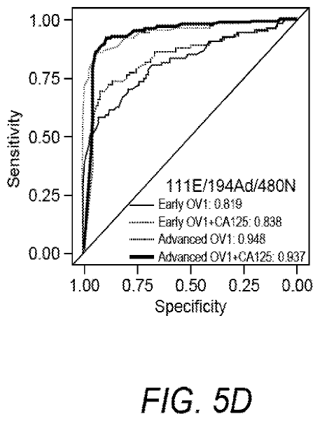

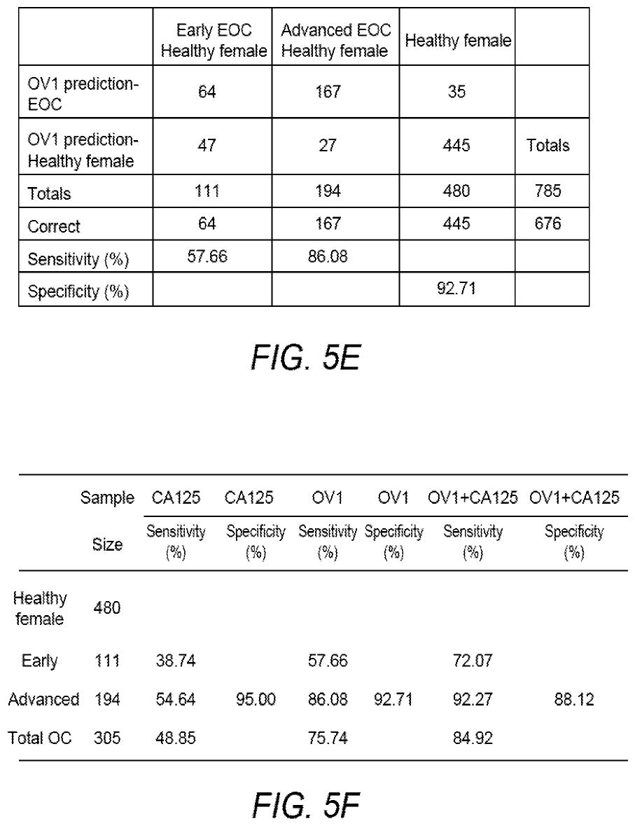

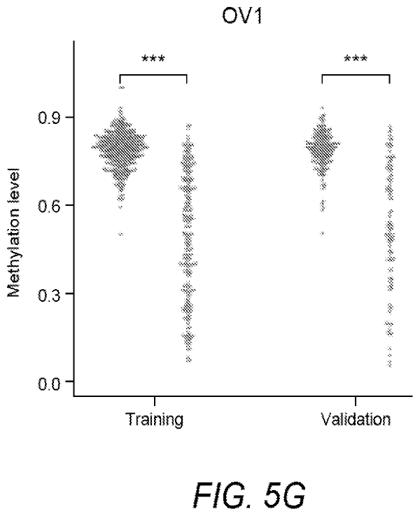

Results are shown in FIGS. 5A to 5E. FIG. 5A shows typical ROC curves of OV1 in the training (red) and validation (blue) datasets of the ddPCR cohort. FIG. 5B provides a Confusion table of binary results of the OV1 prediction model in the training dataset. FIG. 5C provides a Confusion table of binary results of the OV1 prediction model in the validation datasets. FIG. 5D shows typical ROC curves of OV1 and OV1-CA125 combination in distinguishing early (red and green) and advanced (blue and purple) EOC from healthy female in the ddPCR cohort. FIG. 5E provides a Confusion table summarizing OV1 distinguishing early and advanced EOC. FIG. 5F provides a summary of sensitivity and specificity of CA125, OVA-1 ddPCR, and combined CA125 and OVA-1 PCR results in healthy, early EOC, and advanced EOC. FIG. 5G shows Beeswarm plots presenting the methylation levels of OV1 in the ddPCR cohort between ovarian cancer and healthy female, red plots are healthy female samples and blue plots are EOC.

Overall, ddPCR showed a significant methylation difference in OV1 between EOC and healthy female samples (FIG. 5G) hence this cohort was randomly split as 2:1 ratio to training and validation datasets, logistic regression was applied to the training dataset for threshold value determination. As a result, OV1 achieved a 77.4% sensitivity on 92.59% specificity (AUC=0.912) in training dataset and 72.16% sensitivity on 92.95% specificity in validation dataset (AUC=0.877) (FIGS. 5A to 5C). In contrast, CA125 only showed 48.85% sensitivity on 95% specificity in this ddPCR cohort. For diagnosis of different staged EOC of this cohort, OV1 and CA125 achieved 57.66% sensitivity on 92.71% specificity, and 38.74% sensitivity on 95% specificity, respectively in early EOC (n=111), and 86.08% sensitivity on 92.71% specificity, and 54.64% sensitivity on 95% specificity, respectively in advanced EOC (n=194) (FIG. 5D and FIG. 5F, S13). Though the diagnostic sensitivity for early EOC was not satisfactory by neither OV1 nor CA125, OV1 and CA125 in combination dramatically improved the sensitivity to 72.07% while the specificity was still as high as 88.12% (FIG. 5G).

More importantly, when evaluating different sensitivity values (80%, 85%, 90%, 95%) of OV1, Inventors found a very good consistency of corresponding specificity values between the ddPCR assay and the sequencing results (Table 12 and 13), indicating an excellent performance of ddPCR and this platform could be an ideal substitute for sequencing strategy.

TABLE 12
Specificities of OV1 on their corresponding sensitivities (~80%, ~85%, ~90%, ~95%)
in distinguishing EOC from healthy female in the individual cohort.

| | Sample | Sensitivity | Specificity | Sensitivity | Specificity | Sensitivity | Specificity | Sensitivity | Specificity |
|---|---|---|---|---|---|---|---|---|---|
| | size | (%) | (%) | (%) | (%) | (%) | (%) | (%) | (%) |
| Overall | 754 | 80.64 | 82.29 | 84.75 | 74.06 | 90.05 | 61.00 | 94.96 | 35.78 |
| Early | 205 | 65.37 | | 72.20 | | 81.46 | | 90.24 | |
| Stage | | | | | | | | | |
| Advanced | 549 | 86.34 | | 89.44 | | 93.26 | | 96.72 | |
| Stage | | | | | | | | | |

TABLE 13
Specificities of OV1 on their corresponding sensitivities (~80%, ~85%, ~90%, ~95%)
in distinguishing EOC from healthy female in the ddPCR cohort.

| | Sample | Sensitivity | Specificity | Sensitivity | Specificity | Sensitivity | Specificity | Sensitivity | Specificity |
|---|---|---|---|---|---|---|---|---|---|
| | size | (%) | (%) | (%) | (%) | (%) | (%) | (%) | (%) |
| Overall | 305 | 80.00 | 83.33 | 84.92 | 76.46 | 90.16 | 62.92 | 95.08 | 40.21 |
| Early | 111 | 63.96 | | 71.17 | | 81.08 | | 91.00 | |
| Stage | | | | | | | | | |
| Advanced | 194 | 89.69 | | 92.78 | | 95.36 | | 97.42 | |
| Stage | | | | | | | | | |

Inventors also evaluated the utility of OV1 as a methylation marker in a longitudinal cancer screening cohort which consisted of 2117 EOC high-risk participants. OV1 values measured by a ddPCR test were used as the first line tests for this high-risk EOC cohort. Inventors also measured CA125 values. Study participants who received an EOC positive prediction score (the first line test) from the OV1 and CA125 combined model underwent a TVU imaging study (the second line test) conducted by experienced senior sonographers. If no adnexal mass was seen, the participant would take two more TVU tests to verify the result over the following 6 months. If a mass was found, the participant would take a TVU test each month within the next 6 months. Any participant with any suspicion of malignancy or an inconclusive test on a TVU judged by senior sonographers based on subjective assessment would undergo an abdominal MRI for a further study, in which the positive participants went on to have a biopsy for histological confirmation.

In this study, Inventors identified 314 EOC positive participants from the first line tests and confirmed 4 of them to be EOC, 3 of which were at stage I and lof which was at stage III. In addition, after over 18 months' follow-up on this cohort, where each participant took at least two TVU tests annually during these 18 months, only one of the negatively predicted participants were reported with EOC. Therefore, an OV1 and CA125 combined approach gave a sensitivity of 80.0% and a specificity of 85.3% in this prospective cohort, highly consistent with the results in out retrospective ddPCR study cohort.

The global cancer burden lies mainly in late detection. This is particularly true for EOC where the mortality rate is high when detected at the later stages. Reduction in the mortality burden in EOC sufferers relies heavily on early detection. Genetic and epigenetic analysis of cfDNA obtained from liquid biopsies is a promising approach to obtain diagnostic information from just a blood sample. ctDNA can be shed by tumor cells, and importantly they retain the same copy number alterations, mutations and epigenetic markers. Therefore, genetic analysis of these cfDNA could detect early epigenetic changes correlated with malignant transformation. Promising results have been emerging for the use of cfDNA in the diagnosis of different cancers. Conventional search strategies for cancer cfDNA markers utilizes normal and cancer tissue samples or cell lines, comparing mutations and/or epigenetic differences.

Traditional CA125 biomarker is an effective indicator of EOC, our individual and ddPCR cohorts revealed its over 50% sensitivity in all stage EOC and nearly 40% sensitivity in early EOC detection. In fact, an over 200,000 participants' clinical trial has shown that annual CA125 measurement increased early EOC incidence by 39.2% and decreased advanced EOC incidence by 10.2%. However, this performance is still not efficient enough for an ideally first line method for general population screening. Indeed, the EOC related mortality was not significantly reduced in the clinical trial. Achieving a mortality reduction will require more sensitivity screening strategy. Meanwhile, this same trial also indicated that annual TVU is not a good first line screening strategy despite its accuracy since it gave worse than CA125 performance in early EOC detection (23). Intriguingly, in the clinical trial, CA125 increased early EOC incidence by 39.2%, which figure is close to the sensitivity of CA125 in detecting early EOC utilizing in individual and OV-1 ddPCR cohorts (44.2% and 38.7% respectively). If the ˜40% increase in early EOC incidence in the trial is achieved from the ˜40% sensitivity of CA125 in early EOC, then CA125 in combination with our OCPC would increase this early incidence to over 80%. All in all, OCDPs of the inventive concept can be an excellent substitute or supplement for CA125 testing, as they provide increased early EOC diagnostic sensitivity to over 80% while the specificity was as high as 86%.

While large databases are available for mutation markers, that for epigenetic markers are limited in terms of size. Most of the data in epigenetic databases were generated by Infinium® HumanMethylation27 or 450 and most on tumor tissues, which covered less than 0.03% of the human methylome. In this study, Inventors used the TMC-EPIC® Kit, which allowed evaluation of the methylation status of nearly 8 times more loci and regions compared. Indeed, methods of the inventive concept revealed a number of novel EOC-related DMLs and DMRs, based on them, EOC diagnostic and prognostic prediction models were developed which demonstrated superior performance in not only advanced but also early EOC. Nevertheless, the TMC-EPIC® Kit covers less than 1% of the whole methylome, further expanding CpG candidate sites should have great potential to improve EOC screening.

Three characteristics define the ideal EOC diagnostic test: high sensitivity, high PPV and low FPR. A highly sensitive EOC diagnostic test, particularly in the early EOC stage domain will lead to an improvement in cancer mortality. Computer simulations have suggested that improving on the EOC detection sensitivity, currently relying on CA125, could reduce overall mortality by up to 25%. An EOC test with high PPV will help alleviate the anxiety on the patient while waiting for confirmatory TVU results. Finally, a test with low FPR will be a true benefit to healthcare systems because the number of unnecessary TVU tests will be kept to a minimum. The sensitivity, PPV and FPR of an OCDP of the inventive concept were estimated to be 85.81%, 0.84, 0.11 in the individual cohort that composed of 754 EOC patients and 1118 healthy female, while the sensitivity was increased by over 30% compared to CA125 in the same cohort, PPV and FPR were at an acceptable level. Moreover, combining an OCDP of the inventive concept with CA125 can dramatically increase the sensitivity and PPV to be 92.61% and 0.93, respectively, though FPR was compromisingly increased a little to 0.13.

DNA methylation is known to associate with gene expression, methylation at gene promoter in general results in repressed gene expression, whereas function of gene body methylation is elusive. Both over- and down-regulated gene expression were reported in methylated gene bodies. Despite the regulatory relationship, abnormal methylation status generally reflects dysregulated gene expression. In an OCDP of the inventive concept OV18 is annotated to locate at the promoter of TRIM15, which gene was hypomethylated in EOC cfDNA and overexpressed in ovarian cancer tissue. In addition, most of other OCDP markers on gene bodies are associated with dysregulated gene expression, supporting a model in which these markers were tumor sourced. Another interesting finding of this research is that the most powerful prediction marker OV1 is located at intergenic region, mapped to MIR3681HG, a microRNA host gene. Since its cfDNA exhibited over 20% methylation difference between EOC and healthy female cfDNA, indicating that (from the Inventors experience) it its related a gene expression difference of over three-fold. If it is a source of microRNA, its effect on the target DNA would be further enlarged. However, none of research were seen for MIR3681HG and only few predicted targets could be found for MIR3681.

### EXAMPLES

The studies described herein were approved by the Research Ethics Committee of Guangzhou Women and Children's Medical Center and the prospective study was approved by the Research Ethics Committee of the Zhuhai People's Hospital. Inventors studied three retrospective cohorts of EOC and healthy female plasma samples: the pool cohort, the individual cohort and the ddPCR cohort. The pool cohort were composed by pool samples each mixed by >20 individual samples and the other two cohorts were composed by individual samples. In addition, 96 independent plasma samples of endometriosis patients and a prospective cohort of >2000 plasma samples were also included in this investigation.

cfDNA methylation markers were primarily screened in the pool cohort using the TMC-EPIC® Kit for over 3.3 million CpG positions. In this way, 500 markers were selected based on their methylation differences and P-values, these markers were further screened in the individual cohort and seven EOC and an ovarian epithelial cell lines by using their customized probes, the retained markers were employed for the OCDP and OCPP construction in the individual cohort, the best marker OV1 was further assayed on ddPCR platform in the ddPCR cohort. Lastly, OV1 was examined on ddPCR platform in the prospective cohort, and the result were confirmed by imaging. 1.5-2 mL plasma were collected from each subject and stored in −80° C. before the cfDNA were extracted.

### EOC Screening

Inventors conducted a prospective EOC screening cohort study to evaluate the utility of OV1 methylation marker in comparison to a conventional screening method. All the participants were enrolled due to an increased EOC risk, including: (i) female, (ii) post-menopausal. (iii) history of breast cancer or family history of cancers, (iv) BRCA1/2 mutations.

For the high-risk prospective cohort, 2117 subjects were screened by OV1 ddPCR and CA125 as the first line tests, samples predicted positive by the OV1 and CA125 combined model were further tested by TVU as the second line test. Any positive or suspicious TVU finding was followed by an abdominal MRI imaging validation. The validated participants were sent to gynecologists and followed serially, and their tissue samples were obtained and sent to pathologists for histologic confirmation.

### Cell-Free DNA Extraction

Cell-free DNA was isolated from plasma by using the Magen cfDNA extraction Kit® (D3182-04) following the manufacturer's instructions. The quantity of cfDNA was determined by Qubit 2.0® fluorometer (Invitrogen, Life Technologies) with the Qubit dsDNA High Sensitivity Kit® (Invitrogen).

### TMC EPIC Methylation Library Preparing and Sequencing

20 cfDNA samples of EOC at similar stage (early or advanced stage) were pooled together. A Qubit 2.0® fluorometer (Invitrogen, Life Technologies) was employed to estimate the DNA amount of the pool. If the total amount was over 500 ng, the pool was applied to the subsequent library construction. If the amount was less than 500 ng, more cfDNA samples were added to the pool until 500 ng was achieved. cfDNA pools of healthy female were obtained in the same way.

Each pool's methylation library was prepared by using TruSeq Methyl Capture EPIC Library Prep Kit® (FC-151-1003, Illumina), according to the manufacturer's instruction except the fragmentation step were not performed. Concentrations of prepared libraries were determined using the Qubit 2.0® fluorometer (Invitrogen, Life Technologies) and libraries' quality was assessed by capillary electrophoresis (Qseg100®, Bioptic). Qualified libraries were sequenced on the Illumina HiSeq X10® platform (Illumina).

Targeted EPIC Library with Customized Probes

cfDNA extracted from plasma sample were ligated to methylation adaptors by using NEBNext Ultra™ II DNA Library Prep Kit® for Illumina (NEB #7645L) from NEB. The methylation adaptors were composed of an 8-bp index and an 8-bp index linked to a 9-bp UMI sequence, which were customized from Integrated DNA Technologies (reference number: 04099708Q). Adaptor ligated cfDNA were 12-to-1 mixed and hybridized with the customized probes (Integrated DNA Technologies) by using xGen hybridization capture of DNA libraries Kit® (Integrated DNA Technologies). Hybridized mixture samples were eluted by adopting the reagents and steps of the “Second Elution” part of TruSeq Methyl Capture EPIC Library Prep Kit® (FC-151-1003, Illumina), then bisulfite converted by using EZ-96 DNA Methylation-Lightning Mag Prep Kit® (D5047, Zymo Research). Bisulfate converted samples were amplified by adopting the reagents and steps of the “Amplify Enriched Library” part of TruSeq Methyl Capture EPIC Library Prep Kit (FC-151-1003, Illumina). Concentration of prepared libraries were determined using a Qubit 2.0® fluorometer (Invitrogen, Life Technologies) and libraries' quality was assessed by capillary electrophoresis (Qseq100®, Bioptic). Qualified libraries were sequenced on Illumina Nova-seq® platform (Illumina).

### Sequencing Data Analysis

For the pool cohort, raw methylation data were preprocessed using Fastp (version 0.20.0) with default parameters. Clean reads were then aligned to human genome build hg19 using BitmapperBS® (version 1.0.2.3) with default parameters, and bam format results were sorted by Sambamba® (version 0.7.0). DNA methylation calling was performed using MethylDackel® (version 0.4.0) extract default parameter, and DNA methylation calls for methylated and unmethylated controls were extracted from the alignment file. The methylated values located in target regions were extracted using Bedtools® (version 2.29.0).

For the individual cohort, raw methylation data were processed by Umi-tools® (version 1.0.1) with the extract program, and the reads were preprocessed using Fastp® (version 0.20.0) with default parameters. Clean reads were then aligned to human genome build hg19 using BitmapperBS® (version 1.0.2.3) in “pbat” mode, and bam format results were sorted by Sambamba® (version 0.7.0). Aligned reads were deduplicated based on UMIs using umi-tools dedup program. DNA methylation calling was performed using MethylDackel® (version 0.4.0) extract with “—keepDups” parameter, and DNA methylation calls for methylated and unmethylated controls were extracted from the alignment file. The methylated values located in target regions were extracted using Bedtools® (version 2.29.0).

### Statistical Analysis

For both TMC EPIC and targeted EPIC methylation sequencing, differentially methylated CpGs between healthy and tumor samples were identified with DMRfinder® (version 0.3) with the beta-binomial hierarchical modeling and Wald test with the significant cutoff of p<0.05 and 1 Dif1>0.1. ROC analyses were conducted by pROC package to for the assessment of the diagnostic performance. The cd-score between clinical characteristics was evaluated by Wilcoxon rand-sum test and a p-value of <0.05 was considered statistically significant.

### Diagnostic Models

### MethylBERT EOC Model

Large scale unlabeled data for pretraining: To facilitate the pretraining of MethylBERT, we collected extensive DNA methylation data from two primary sources: the GEO-methyl dataset and the TCGA-methyl dataset. In total, we amassed over 110,000 samples, with the data exceeding 3 terabytes in size. Both datasets contain comprehensive genome-wide methylation data from diverse tissue types and conditions, providing a rich resource for training and evaluating the model's performance.

The GEO-methyl dataset is derived from the National Center for Biotechnology Information (NCBI) Gene Expression Omnibus (GEO) repository, which houses a large collection of high-throughput sequencing and microarray datasets. Inventors focused on whole-genome bisulfite sequencing (WGBS) and reduced representation bisulfite sequencing (RRBS) datasets that provide genome-wide methylation data across various tissues, diseases, and conditions. We retrieved Accession data from several platforms, including GPL13534, GPL21145, GPL8490, GPL23976, and GPL9183, and extracted beta values files for further analysis. After data cleaning and preprocessing, the GEO-methyl dataset comprises methylation data from 95,995 samples, covering a wide range of species, including humans, mice, and plants. This dataset offers a diverse and extensive resource for pretraining MethylBERT model.

The TCGA-methyl dataset is derived from The Cancer Genome Atlas (TCGA), a comprehensive resource containing multi-omics data for over different cancer types. For MethylBERT, Inventors focused on the DNA methylation data generated using the Illumina Infinium HumanMethylation450 BeadChip® platform. The TCGA-methyl dataset includes methylation data from 15,439 human samples, comprising both tumor and adjacent normal tissues. By incorporating this dataset, MethylBERT was enabled to learn the diverse methylation patterns associated with various cancer types and stages.

CpG Site Representation: MethylBERT features an innovative CpG site embedding scheme, comprising four distinct embedding types: chromosome embedding (E_{c}), position embedding (Eₚ), methylation level embedding (Eₗ), and gene embedding (E_{g}). Each embedding captures unique aspects of CpG site information, enhancing the model's performance.

Chromosome embedding E_{c}: By representing each CpG site's chromosome as an embedding, the model can learn and differentiate among various chromosomal contexts, taking into account functional and structural variations across chromosomes.

Position embedding Eₚ: CpG sites are assigned to bins, each L base pairs (bps) in length, with a unique embedding assigned to each bin. This allows the model to learn relationships between neighboring CpG sites and capture the spatial organization of methylation patterns within the genomic landscape. In this implementation, L is set to 2000.

Methylation level embedding Eᵢ: To facilitate the learning of methylation patterns, continuous methylation levels, which range from 0 to 1, are discretized into B bins. This approach enables the model to effectively capture the nuances of methylation dynamics. In this case, B is set to 20. Gene embedding E_{g}: Gene embeddings are employed for CpG sites to associate them with their potential functional roles. For sites with known correlations, the closest gene is used; for those without, the nearest downstream gene is selected. These gene embeddings are derived from gene2vec, which learns gene-gene association information based on gene expression profiles across various tissues and conditions, enabling the creation of gene-gene co-expression networks. The final CpG site embedding is obtained by summing these embeddings: E=E_{c}+Eₚ+Eₜ+E_{g}.

Neural Network Architecture: The attention mechanism of the Transformer architecture exhibits quadratic computational complexity, posing a significant challenge when handling more than 20 million CpG sites. To address this issue, Inventors employed the Performer, a matrix decomposition-based Transformer model designed to reduce computational complexity from quadratic to linear (O(L₂) to O(L), enabling efficient processing of large-scale data. The Performer utilizes an approximation technique termed “kernelized attention” with random feature maps. In contrast to the standard Transformer attention, represented as Att(Q, K, V)=softmax(QK_{T} squrt(dₖ))V, the Performer attention mechanism is formulated as Att(Q, K, V)=softmax(ΦQ)Φ(K)^{T}/sqrt(dₖ)V. Here, Q, K, and V denote the query, key, and value matrices, respectively, and Φ signifies a feature map function that projects input into a new space, facilitating efficient approximation of the dot-product attention. In this study, the number of Transformer layers is set to six.

Pretraining with Masked Methylation Level Prediction: In the pretraining phase, Inventors adapt the masked language model (MLM) objective, used in BERT, to suit the methylation data to generate a masked methylation level prediction (MMLP). The goal of MMLP is to predict the methylation level of some masked CpG sites, given the context of their surrounding CpG sites. To achieve this, a certain percentage Pₘ of CpG sites in the input sequence is masked, and the model is trained to predict their methylation levels. Following BERT, we set ft. to 15%. Formally, let x, be the input CpG sequence, where i∈1, . . . , L, and yₜ be the corresponding ground truth methylation levels. During pretraining, we randomly mask P of CpG sites, replacing their methylation level embeddings with a special [MASK] token. The MMLP loss is calculated as the cross-entropy between the predicted methylation levels p, and the ground truth yₜ for the masked positions: MMLP Loss Lₘₐₛₖ=−Σ_{i∈M}*yₜ log(ŷₜ), where ŷₜ=softmax(f,xᵢ)), M is a set of CpGs with masked methylation levels, and f(xᵢ) is the output of MethylBERT model for the masked CpG site i. The model is optimized to minimize this loss function, encouraging it to learn biologically relevant patterns and correlations between CpG sites.

To ensure computational efficiency and stability during the training process, Inventors developed two strategies. First, Inventors segmented the data chromosome-wise, allowing the model to concentrate on smaller, more manageable portions of the data while preserving the unique characteristics of each chromosome. By training on individual chromosome sections, the model can learn essential features and relationships specific to each genomic region. Second, Inventors implemented a random down sampling strategy, selecting N CpG sites per training sample. Initially, a contiguous set of N CpG sites is chosen, with Nₘ ranging from N to 10N. Subsequently, N CpG sites are randomly selected from this set. This method ensures a representative subset of CpG sites is obtained, capturing relevant information while maintaining computational efficiency. In this study, N is set to 8192. By adopting these strategies, MethylBERT effectively addresses the computational challenges posed by the large-scale nature of DNA methylation data, enabling the model to learn meaningful CpG site representations and relationships without compromising performance or scalability.

Supervised Learning for EOC Detection: Inventors fine-tuned the pretrained MethylBERT model for EOC detection tasks. Inventors first employed MethylBERT to obtain representations for each of all chromosomes, then concatenate these representations to form a sample representation, which can be used for the final prediction. For a given sample, let hₑ denote the representation of chromosome e obtained from MethylBERT, where c∈{1, . . . , C}. We concatenate these representations to form the final sample representation z=Concat(h₁, h₂, . . . , h_{C}). Here, for human samples C is 23. In the training dataset of 503 EOC and 744 healthy female samples that were randomly selected from the individual cohort, to predict the presence or absence of EOC in a sample, we employ a binary classification approach using a fully connected layer followed by a sigmoid activation function, obtaining the probability ŷᵢ of the i-th sample belonging to the EOC class (y=1). The model is trained to minimize the binary cross-entropy loss L_{binary}(ŷᵢ, yᵢ), where yᵢ denotes the ground truth label. By fine-tuning MethylBERT model in a supervised manner for EOC detection, we enabled it to capture EOC-specific methylation patterns and relationships, ultimately resulting in MethylBERT-EOC diagnostic model for detecting EOC in DNA methylation data.

### LASSO-EOC Model

Samples in training and validation datasets of the individual cohort were as same as which were used for MethylBERT-EOC diagnostic model construction. The 493 markers screened-out from pool cohort were processed by LASSO in the training dataset to distinguish EOC from healthy female samples. 500 times of LASSO were performed with each time randomly selecting 70% of samples. Markers that appeared in over 450 times of LASSO were retained and were applied to the training dataset to construct an EOC diagnostic model based on logistic regression, then the diagnostic model was tested in the validation dataset.

### Prognostic Model

EOC samples with complete survival information in the individual cohort were randomly split with 2:1 ratio to training and validation datasets. Markers were pre-screened in the pool cohort and cell lines. The screened-out markers were processed by LASSO in the training dataset to distinguish samples of incidence from samples of other observations. 100 rounds of LASSO were performed with each round randomly selecting 70% samples. Markers appeared in over 90 rounds of LASSO were retained. Concurrently, Unicox was also employed to process the selected markers in the training dataset to distinguish samples of incidence from samples of other observations. Markers with P-value <0.05 in Unicox were overlapped with the markers retained from LASSO. These overlapped markers were applied to the training dataset to construct an EOC prognostic model based on logistic regression, then the prognostic model was tested in the validation dataset.

### Droplet Digital PCR

cfDNA samples were extracted from plasma and bisulfite converted by using the EZ DNA Methylation-Lightning Kit® (Zymo Research, Irvine, CA, USA) according to the manufacturer's instructions. The subsequently examination and analysis was based on the droplet digital PCR system according to the manufacturer's instruction (Bio-Rad, Pleasanton, California, USA). FAM and HEX fluorophore were employed to label methylation probe and unmethylation probe, respectively, and the sequence of probes and primers were indicated in Table 11. For each reaction, the reaction system and parameter were as following:

### PCR Reagent Mixture—

| | |
|---|---|
| 2x ddPCR ™ Supermix for Probes | 10 μl |
| (No dUTP) (Bio-Rad) | |
| primer mix (10 μM) | 1.6 μl |
| probe mix (10 μM) | 0.8 μl |
| bisulfite-converted DNA | 0.6 μl |
| Nuclease free water (AM9937, | 0.6 μl |
| Life Technologies Corp.) | |

### PCR Reaction Conditions—

| | | | |
|---|---|---|---|
| (a) | 98° C. | 10 | min |
| (b) | 98° C. | 30 | s |
| (c) | 45.7° C. | 60 | s |

Repeat step (b) and (c) for 39 rounds

| | | |
|---|---|---|
| (d) | 98° C. | 10 min |
| (e) | 4° C. | 20 min |

### Cell Lines, Cell Culture, and DNA Extraction

Cell lines were obtained from the American Type Culture Collection (ATCC) and maintained according to the supplier's instructions. HO-8910, A2780, and IOSE-80 cells were grown in RPMI-1640 with 10% FBS; SK-OV-3 cells were grown in McCoy's 5A with 10% FBS; OVCAR-3 cells were grown in RPMI-1640 with 20% FBS and 0.01 mg/mL insulin; Hey and Anglne cells were grown in DMEM with 10% FBS; SW626 cells were grown in Leibovitz's L-15 with 10% FBS. Cell DNA was extracted for targeted EPIC methylation sequencing using the QIAamp DNA Mini Kit following the manufacturer's specifications.

### Prospective EOC Screening Cohort Study Design

The prospective study was approved by the Research Ethics Committee of the Zhuhai People's Hospital. We conducted a prospective EOC screening cohort study from August 2022 to July 2023 to evaluate the utility of the OV1 methylation marker in combination with a conventional screening method. All the participants were enrolled due to an increased EOC risk, including (i) female, (ii) post-menopausal, (iii) history of breast cancer or family history of cancers, and (iv) BRCA1/2 mutations.

For the high-risk prospective cohort, 2117 subjects were screened by OV1 ddPCR and CA125 as the first line tests, samples predicted positive by the OV1 and CA125 combined model were further tested by TVU as the second line test. Any positive or suspicious TVU finding was followed by an abdominal MRI imaging validation. The validated participants were sent to gynecologists and followed serially, and their tissue samples were obtained and sent to pathologists for histologic confirmation.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

1. A method of assisting in diagnoses of endothelial ovarian cancer, comprising:
isolating cell-free genetic material from an individual; and
characterizing nucleic acid methylation of one or more genetic markers selected from the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22,
wherein aberrant methylation of one or more genetic marker is indicative of endothelial ovarian cancer.

2. The method of claim 1, wherein the cell-free genetic material is cell-free DNA.

3. The method of claim 1, comprising treating at least a portion of the cell-free genetic material with bisulfite.

4. The method of claim 1, comprising contacting the cell-free genetic material with a nucleic acid primer complementary to a portion of the genetic material proximal to at least one of the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22_and performing an nucleic acid amplification to generate an amplification product.

5. The method of claim 4, comprising contacting the amplification product with a probe that is complementary to at least of the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22.

6. The method of claim 1, comprising characterizing levels of CA125, wherein elevated levels of CA125 are indicative of endothelial ovarian cancer.

7. The method of claim 1, wherein the genetic marker is OV1.

8. A method of assisting in determining a prognosis for endothelial ovarian cancer, comprising:
isolating cell-free genetic material from an individual; and
characterizing nucleic acid methylation of one or more genetic markers selected from the group consisting of OV1, OV2, OV6, OV3, [[ ]]OV4, OV5, OV6, OV6, OV6, OV6, OV7, OV8, OV9, OV10, OV11, OV12, OV13, OV14, OV15, OV16, OV17, OV18, OV19, OV20, OV21, and OV22,
wherein aberrant methylation of one or more genetic marker is indicative of a poor prognosis for endothelial ovarian cancer.

9. The method of claim 8, wherein the cell-free genetic material is cell-free DNA.

10. The method of claim 8, comprising treating at least a portion of the cell-free genetic material with bisulfite.

11. The method of claim 9, comprising contacting the cell-free genetic material with a nucleic acid primer complementary to a portion of the genetic material proximal to at least one of the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22_and performing an nucleic acid amplification to generate an amplification product.

12. The method of claim 11, comprising contacting the amplification product with a probe that is complementary to at least of the group consisting of OV1, OV2, OV6, OV7, OV9, OV11, OV18, and OV22.

13. The method of claim 8, comprising characterizing levels of CA125, wherein elevated levels of CA125 are indicative of a poor prognosis for endothelial ovarian cancer.

14. The method of claim 8, wherein the genetic marker is OV1.

15. The method of claim 1, further comprising, prior to the step of characterizing methylation, applying an artificial intelligence algorithm to a training dataset comprising individuals identified as having EOC and individuals without EOC and identifying a methylation pattern associated with individuals with EOC, wherein the artificial intelligence algorithm comprises correlation with chromosome embedding, position embedding, methylation level embedding, and gene embedding, and wherein the methylation pattern comprises one or more genetic markers exhibiting methylation differences between individuals with EOC and individuals without EOC.

16. The method of claim 15, wherein the artificial intelligence algorithm comprises a matrix decomposition-based Transformer model configured to reduce computational complexity from quadratic to linear (O(L²) to O(L)), thereby enabling efficient processing of large-scale data.

17. The method of claim 16, wherein the Transformer model comprises a Performer, wherein the Performer comprises an attention mechanism formulated as Att(Q, K, V)=softmax(ΦQ)Φ(K)^{T}/sqrt(dₖ)V, wherein Q, K, and V denote query, key, and value matrices, respectively and Φ signifies a feature map function that projects input into a new space, thereby improving efficient approximation of the dot-product attention.

18. A composition for diagnosis or prognosis of endothelial ovarian cancer, comprising:
a first primer that is complementary to a first portion of nucleic acid proximal to at least one genetic marker selected from the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22; and
a probe that is complementary to at least one genetic marker selected from the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22 and that comprises a first dye or a first fluorophore.

19. The composition of claim 18, further comprising a second primer that is complementary to a genetic marker selected from the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22.

20. The composition of claim 19, comprising a second probe that is complementary to a genetic marker selected from the group consisting of OV1, OV2, OV6, OV3, OV4, OV5, OV6, OV6, OV6, OV6, OV7, OV8, OV9, OV10, OV11, OV12, OV13, OV14, OV15, OV16, OV17, OV18, OV19, OV20, OV21, and OV22, optionally, wherein the second probe comprises a second dye or a second fluorophore.

21. (canceled)
